(19) United States
(12) Patent Application Publication
(10) Pub. No.: US 2018/0258123 A1
(43) Pub. Date: Sep. 13, 2018

(54) Suppressors of Premature Termination Codons as Therapeutics and Methods for Their Use

(71) Applicant: The University of British Columbia, Vancouver (CA)

(72) Inventors: Michel Roberge, Vancouver, BC (CA); Alireza Baradaran-Heravi, Vancouver, BC (CA); Carla D. Zimmerman, Vancouver, BC (CA); Aruna Dinesh Balgi, Burnaby, BC (CA); Stephen G. Withers, Vancouver, BC (CA); Kunho Choi, Vancouver, BC (CA)

(21) Appl. No.: 15/762,001

(22) PCT Filed: Sep. 23, 2016
(86) PCT No.: PCT/CA2016/000240

§ 371 Date: Mar. 21, 2018

Related U.S. Application Data
(60) Provisional application No. 62/232,789, filed on Sep. 25, 2015.

(51) Int. Cl. C07H 15/234; A61K 31/7036

(52) U.S. Cl. CPC C07H 15/234; A61K 31/7036

(57) ABSTRACT

This invention discloses the use of aminoglycoside antibiotics such as gentamicin B1 to suppress premature termination codons during translation and promote the full length read-through of transcripts such as p53 that incorporate nonsense mutations and to treat disease conditions such as cancer caused by such genetic mutations.

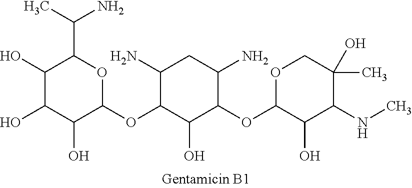 [SMILES: CNC1C(O)C(OC2C(N)CC(N)C(OC3OC(C(C)N)C(O)C(O)C3O)C2O)OCC1(C)O]

### TECHNICAL FIELD

This invention relates to therapeutic compounds and compositions, and methods for their use in the treatment or amelioration of various indications, including medical conditions associated with premature termination codons (PTCs) in RNA, including various cancers. In particular the invention relates to therapies and methods of treatment that would at least partially restore translation of full-length protein products.

### CROSS REFERENCE TO RELATED APPLICATIONS

This application claims the benefit of U.S. Provisional Patent Application Ser. No. 62/232,789 filed 25 Sep. 2015.

### BACKGROUND

Genomics advances will soon make it routine to identify the precise molecular lesions responsible for many of the rare genetic diseases that afflict our population. Unfortunately, most of these diseases have no treatments, in Canada about 30% of patients die in childhood, and it is exceedingly difficult to develop disease-specific treatments because of the small number of patients for each disease and the high cost of developing new drugs. About 10% of disease-causing mutations are nonsense mutations that introduce a PTC.

The European Organization for Rare Diseases estimates that there are at least 5,000 rare genetic diseases, defined as affecting less than 1 in 2,000 people and the genes for about 4,000 have been identified (Online Mendelian Inheritance in Man database). Rare genetic diseases are believed to affect 5-6% of the population, or about 25 million people in the EU, 16 million in the USA and 1.8 million in Canada. It is estimated that 95% of rare genetic diseases have no specific treatment. Furthermore, genetic diseases that would not have the rare classification, also have nonsense mutations. It is estimated that 20.3% of the ˜43,000 disease-associated single-base pair substitutions affecting gene coding regions that are cataloged in the Human Gene Mutation Database (HGMD 2007-Mort M. et al. 2008) are PTCs.

For nearly all these diseases, about 10-11% of patients have nonsense point mutations. These mutations change an amino acid codon to a PTC (i.e. UAA, UAG and UGA). PTCs may result in decreased mRNA stability via nonsense-mediated mRNA decay (NMD), as well as production of some truncated non-functional protein, if any protein is produced. Compounds that allow insertion of an amino acid at a PTC, without affecting normal termination codons, can enable production of functional full-length protein. This approach, termed both nonsense mutation suppression and PTC read-through, offers the possibility of developing a single treatment for large numbers of patients across multiple diseases. In reality, a proportion of these patients would likely not benefit from such a therapy, for example those children born with irreversible neurological damage. Nevertheless, for 50% of rare genetic diseases, the onset of disease occurs in childhood and progressively worsens, and these patients are the ones who stand to benefit most from nonsense suppression therapy.

The therapeutic potential of nonsense suppression is not limited to inherited disorders. Nonsense mutations also occur in tumour suppressor genes in about 10% of cases of sporadic cancer, which affects 40% of the population and is far from rare. To illustrate, the R213X mutation in protein p53 is present in 1% of all human cancers. This corresponds to about 220,000 cases worldwide (Hoe, K. K. Verma, C. S. and Lane, D. P. 2014) that could theoretically benefit from nonsense suppression therapy. A further 70 cancer-driver tumour suppressor genes have been identified (Vogelstein B, et al. 2013). Tumour sequencing and mutation analysis is not yet routine for cancer diagnosis. However, the concept of personalized medicine has taken huge steps in the cancer field and it is anticipated that identifying nonsense mutations in cancer will become routine in the next decade.

Accordingly, the targeting of nonsense mutations could eliminate the “rare” element of rare genetic diseases in some cases where the genetic disease is caused at least in part by a nonsense mutation and nonsense suppression may also be of use in the treatment of some cancers.

Compounds that enable PTC read-through, offer the possibility of using the same treatment for large numbers of patients across multiple diseases based on the mechanism of the PTC and not the particular gene having the PTC.

High concentrations of aminoglycoside antibiotics were shown 30 years ago to induce PTC read-through in some yeast genes (Singh A et al. 1979) and in a reporter gene in mammalian cells (Burke J F and Mogg A E. 1985). The potential for using gentamicin to treat cystic fibrosis patients with a PTC in the CFTR gene was shown when gentamicin was used to induce CFTR protein expression from the endogenous gene in a patient-derived bronchial epithelial cell line (Bedwell D M et al. 1997), recovery of function in mice bearing the human CFTR G542X transgene (Du M et al. 2002) and increases in CFTR chloride conductance in patients (Clancy J P et al. 2001; and Wilschanski M et al. 2003). Similarly, paromomycin, geneticin (G418) and PTC124 (3-[5-(2-fluorophenyl)-[1,2,4]oxadiazole-3-yl]-benzoic acid) are all reported to have nonsense suppressive properties (Karijolich J, and Yu, Y-T 2014). In all cases the improvement was small and patient response was variable (Linde L et al. 2007). The lack of potency, the recognized renal and otic toxicities of high dose gentamicin and the need for intravenous or intramuscular administration likely limited its further development.

Read-through by gentamicin was demonstrated in mdx mice (Barton-Davis E R et al. 1999) with a PTC introduced into the mouse dystrophin gene to model human Duchenne Muscular Dystrophy (DMD). The first small trial in DMD patients showed no effect of gentamicin. Two others showed dystrophin expression in some patients (Malik V et al. 2010) but the level of expression was insufficient for patient improvement. Again, dose-limiting toxicities prevented further development.

Major efforts have been put into developing aminoglycoside derivatives with reduced toxicity e.g. (Shulman E et al. 2014; and Xue X et al. 2014) and discovering non-aminoglycoside RT compounds such as RTC13, RTC14, GJ71, GJ72 and PTC124 (Gatti R A. 2012; and Welch E M et al. 2007). These compounds increased protein production in several cell culture and animal disease models, but often at the limit of detection by western blotting for endogenous gene expression and with variable responses between genes, cell lines, and PTC mutations.

Furthermore, there are numerous approaches to read-through therapy. For example, read-through drugs, suppressor tRNAs, PTC pseudouridylation, and inhibition of nonsense-mediated mRNA decay (Keeling, K. M. et al. 2104).

PTC124 (Translarna™) is the sole new compound to have entered clinical trials. It is orally bioavailable and has a good safety profile compared with aminoglycosides. PTC124's PTC RT activity has been challenged based on artifactual activity in luciferase reporter assays of the type used for its discovery and lack of demonstrable RT activity in other reporter assays (McElroy S P et al. 2013). Nevertheless, it has shown activity in higher model systems, including increased dystrophin expression and muscle function in the mdx mouse (Welch E M et al. 2007) and CFTR protein expression and improved chloride conductance in the intestine of the G542X-hCFTR mouse (Du M et al. 2008). Recently, a phase 3 clinical trial in CFTR (Kerem E. 2014) and a phase 2b trial in DMD patients (Bushby K et al. 2014) both failed to reach statistical significance. However, retrospective analyses hinted at signs of efficacy in subgroups of authorization for DMD treatment in the European Union, conditional upon completion of a phase 3 trial (mid-2015) and submission of additional safety and efficacy data (Ryan N J. 2014).

Overall, currently available RT compounds suffer two major limitations: they display low activity, typically inducing less than 5% of wild-type (wt) protein levels; and they show unpredictable activity in only a small subset of genetic disease systems tested.

### SUMMARY

This invention is based in part on the discovery that compounds described herein suppress premature termination codons. Specifically, compounds identified herein, show the ability to read through premature stop codons.

In accordance with one embodiment, there is provided a pharmaceutical composition including 1) a compound, or a pharmaceutically acceptable salt thereof, in an amount effective for treating or ameliorating a medical condition associated with premature termination codons (PTCs) in RNA, wherein the compound has the structure of Formula II:

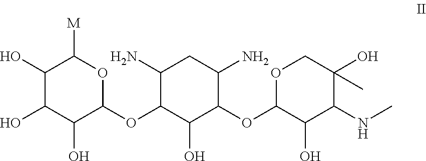

wherein M may be

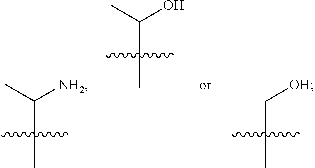

and 2) a pharmaceutically acceptable excipient or pharmaceutically acceptable carrier.

In accordance with a further embodiment, there is provided a pharmaceutical composition including i) a compound, or a pharmaceutically acceptable salt thereof, in an amount effective for treating or ameliorating a medical condition associated with premature termination codons (PTCs) in RNA, wherein the compound has the structure of Formula I:

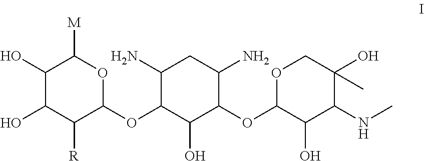

wherein R may be OH or NH₂;

M may be

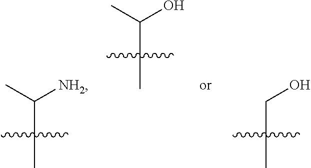

when R is OH and M may be

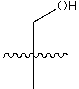 [SMILES: *CO]

when R is NH₂; and 2) a pharmaceutically acceptable excipient or pharmaceutically acceptable carrier.

In accordance with a further embodiment, there is provided a method of treating or ameliorating a medical condition associated with premature termination codons (PTCs) in RNA, the method including administering a compound, or a pharmaceutically acceptable salt thereof, in an amount effective for treating or ameliorating a medical condition associated with a PTC in RNA, wherein the compound has the structure of Formula II:

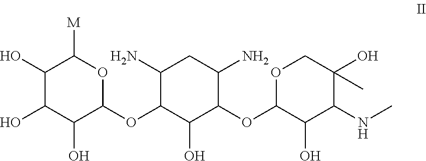

wherein M may be

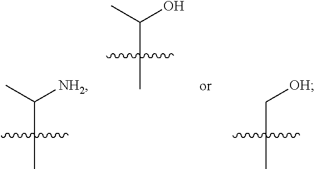

to a subject in need thereof. Alternatively, the method may have a compound of Formula I.

In accordance with a further embodiment, there is provided a method of promoting read-through of a premature termination codon (PTC) in a RNA sequence, the method including administering a compound, or a pharmaceutically acceptable salt thereof, in an amount effective for treating or ameliorating a medical condition associated with a PTC in RNA, wherein the compound has the structure of Formula II:

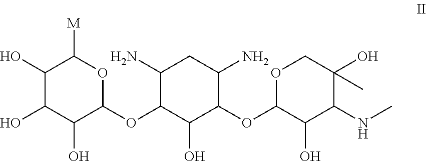

wherein M may be

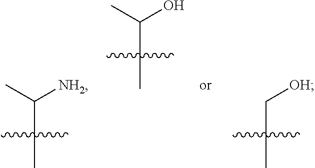

to a subject in need thereof. Alternatively, the compound may be of Formula I.

In accordance with a further embodiment, there is provided a method of promoting production of a functional protein in a cell, the protein encoded by a nucleotide sequence comprising a premature termination codon (PTC), the method comprising contacting the cell with an effective amount of a compound having the structure of Formula II:

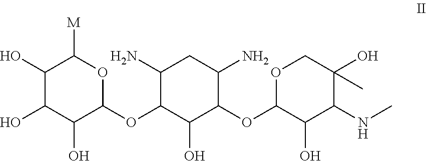

wherein M may be

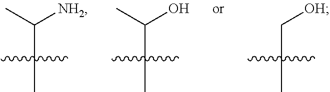

to a subject in need thereof. Alternatively, the compound may be of Formula I.

In accordance with a further embodiment, there is provided a compound, wherein the compound has the structure:

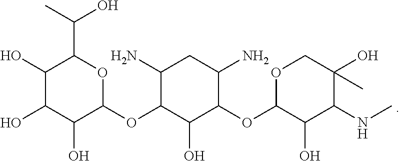 [SMILES: CNC1C(O)C(OC2C(N)CC(N)C(OC3OC(C(C)O)C(O)C(O)C3O)C2O)OCC1(C)O]

In accordance with a further embodiment, there is provided a pharmaceutical composition, the pharmaceutical composition comprising: a compound having the structure

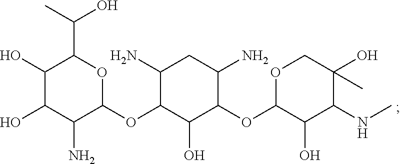 [SMILES: CNC1C(O)C(OC2C(N)CC(N)C(OC3OC(C(C)O)C(O)C(O)C3N)C2O)OCC1(C)O]

and a steroid.

In accordance with a further embodiment, there is provided a method of treating or ameliorating a medical condition associated with premature termination codons (PTCs) in RNA, the method including administering a compound, or a pharmaceutically acceptable salt thereof, in an amount effective to treat or ameliorate a medical condition associated with a PTC in RNA, wherein the compound has the structure of

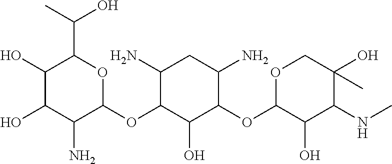 [SMILES: CNC1C(O)C(OC2C(N)CC(N)C(OC3OC(C(C)O)C(O)C(O)C3N)C2O)OCC1(C)O]

in combination with a steroid, to a subject in need thereof.

In accordance with a further embodiment, there is provided a use of a compound, or a pharmaceutically acceptable salt thereof, in an amount effective for treating or ameliorating a medical condition associated with premature termination codons (PTCs) in RNA, wherein the compound has the structure of Formula II:

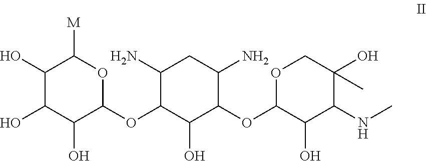

wherein M may be

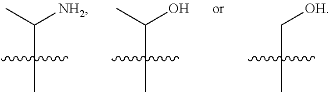

Alternatively, the compound may be of Formula I.

In accordance with a further embodiment, there is provided a use of a compound in the manufacture of a medicament for treatment or amelioration of a medical condition associated with premature termination codons (PTCs) in RNA, wherein the compound has the structure of Formula II:

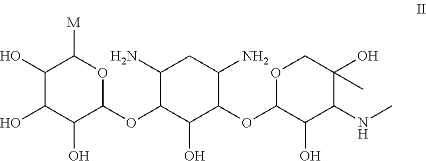

and wherein M may be

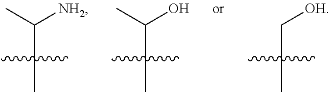

Alternatively, the compound may be of Formula I.

In accordance with a further embodiment, there is provided a commercial package comprising: (a) a compound having the structure of Formula II:

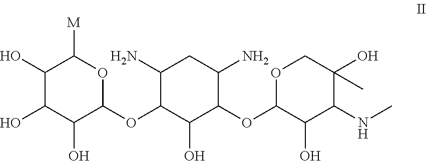

wherein M may be

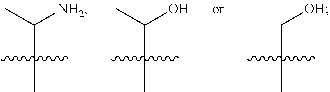

and (b) instructions for treating or ameliorating a medical condition associated with premature termination codons (PTCs) in RNA. Alternatively, the compound ma be of Formula I.

The compound may be selected from one or more of the following:

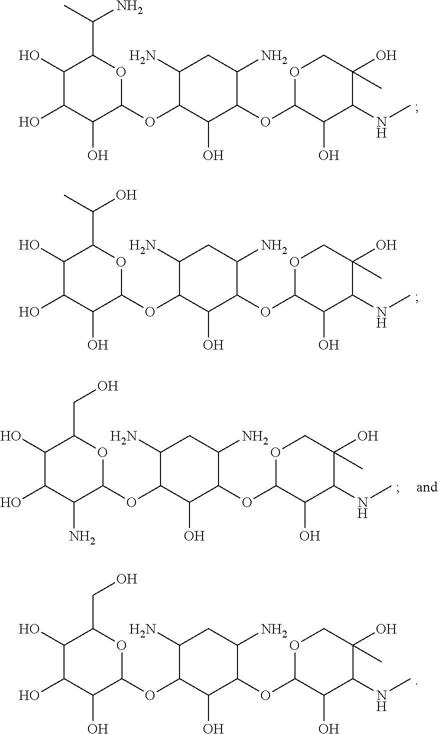

The compound may be selected from one or more of the following:

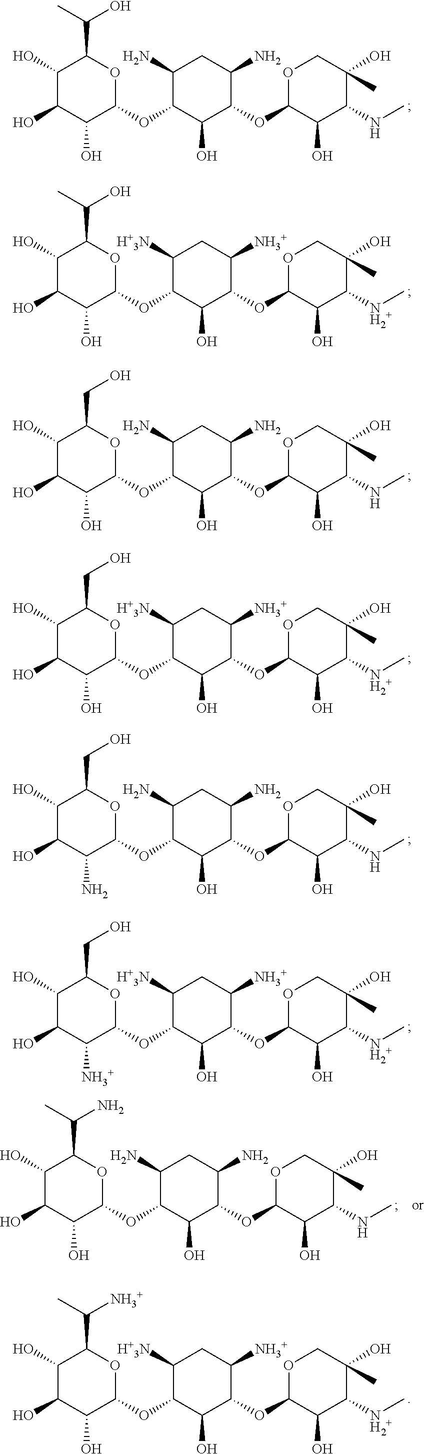

The compound may be selected from one or more of the following:

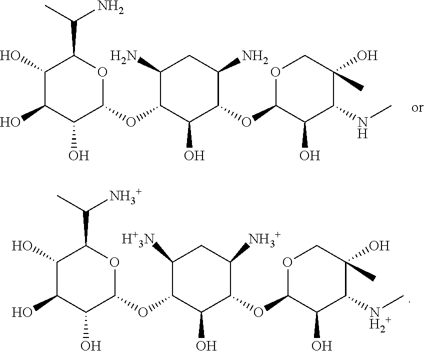

The medical condition may be selected from one or more of the conditions listed in TABLE 1 or TABLE 2. The medical condition may be selected from TABLE 1 or TABLE 2. The medical condition may be selected from TABLE 1. The medical condition may be selected from TABLE 2.

The medical condition may be selected from the group consisting of: central nervous system disease; peripheral nervous system disease; neurodegenerative disease; autoimmune disease; DNA repair disease; inflammatory disease; collagen disease; kidney disease; pulmonary disease; eye disease; cardiovascular disease; blood disease; metabolic disease; neuromuscular diseases; neoplastic disease; and any genetic disorder caused by nonsense mutation(s).

The medical condition may be selected from the group consisting of: ataxia-telangiectasia; muscular dystrophy; Duchenne muscular dystrophy; Dravet syndrome; myotonic dystrophy; multiple sclerosis; infantile neuronal ceroid lipofuscinosis; Alzheimer's disease; Tay-Sachs disease; neural tissue degeneration; Parkinson's disease; chronic rheumatoid arthritis; lupus erythematosus; graft-versus-host disease; primary immunodeficiencies; severe combined immunodeficiency; DNA Ligase IV deficiency; Nijmegen breakage disorders; xeroderma pigmentosum (XP); rheumatoid arthritis; hemophilia; von Willebrand disease; thalassemia (for example; β-thalassemia); familial erythrocytosis; nephrolithiasis; osteogenesis imperfecta; cirrhosis; neurofibroma; bullous disease; lysosomal storage diseases; Hurler's disease; familial cholesterolemia; cerebellar ataxia; tuberous sclerosis; immune deficiency; cystic fibrosis; familial hypercholesterolemia; pigmentary retinopathy; retinitis pigmentosa; amyloidosis; atherosclerosis; giantism; dwarfism; hypothyroidism; hyperthyroidism; aging; obesity; diabetes mellitus; familial polycythemia; Niemann-Pick disease; epidermolysis bullosa; Marfan syndrome; Becker muscular dystrophy (BMD); spinal muscular atrophy; cancer; and any genetic disorder caused by nonsense mutation(s).

The medical condition may be cancer. The cancer may be of the head and neck, eye, skin, mouth, throat, esophagus, chest, bone, blood, lung, colon, sigmoid, rectum, stomach, prostate, breast, ovaries, kidney, liver, pancreas, brain, intestine, heart or adrenals. The cancer may be sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor or multiple myeloma. The cancer may be acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, hairy cell leukemia, or multiple myeloma.

The premature termination codon may be UGA or UAG. The premature termination codon may be UGA. The premature termination codon may be UAG. The premature termination codon may be UAA.

The method may further include the administration of a steroid to the subject. The steroid may be selected from one or more of the following: Medroxyprogesterone; Betamethasone; Dexamethasone; Beclomethasone; Budesonide; Clobetasol propionate; Cortisone acetate; Flumethasone Pivalate; Fluticasone Propionate; Hydrocortisone; Methylprednisolone; Paramethasone; Prednisolone; Prednisone; Triamcinolone; Danazol; Fludrocortisone; Mifepristone; Megestrol acetate; and Progesterone.

The compound may be

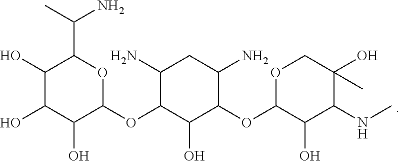 [SMILES: CNC1C(O)C(OC2C(N)CC(N)C(OC3OC(C(C)N)C(O)C(O)C3O)C2O)OCC1(C)O]

The compound may be

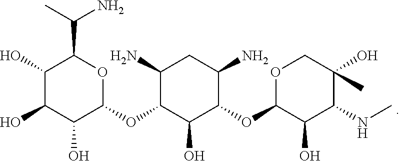 [SMILES: CN[C@@H]1[C@@H](O)[C@@H](O[C@@H]2[C@@H](O)[C@H](O[C@H]3O[C@H](C(C)N)[C@@H](O)[C@H](O)[C@H]3O)[C@@H](N)C[C@H]2N)OC[C@]1(C)O]

The compound may be

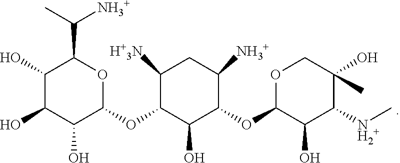

The compound may be

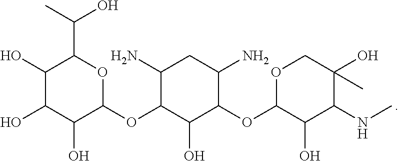 [SMILES: CNC1C(O)C(OC2C(N)CC(N)C(OC3OC(C(C)O)C(O)C(O)C3O)C2O)OCC1(C)O]

The compound may be

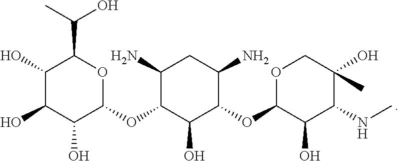 [SMILES: CN[C@@H]1[C@@H](O)[C@@H](O[C@@H]2[C@@H](O)[C@H](O[C@H]3O[C@H](C(C)O)[C@@H](O)[C@H](O)[C@H]3O)[C@@H](N)C[C@H]2N)OC[C@]1(C)O]

The compound may be

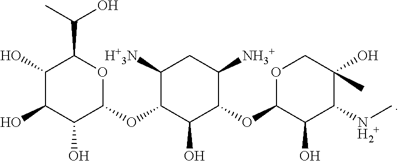

The compound may be

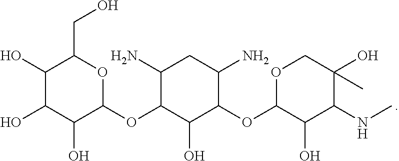 [SMILES: CNC1C(O)C(OC2C(N)CC(N)C(OC3OC(CO)C(O)C(O)C3O)C2O)OCC1(C)O]

The compound may be

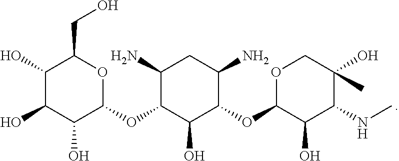 [SMILES: CN[C@@H]1[C@@H](O)[C@@H](O[C@@H]2[C@@H](O)[C@H](O[C@H]3O[C@H](CO)[C@@H](O)[C@H](O)[C@H]3O)[C@@H](N)C[C@H]2N)OC[C@]1(C)O]

The compound may be

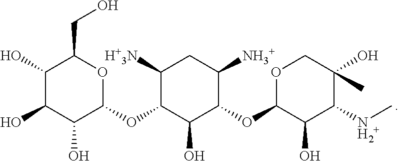

The compound may be

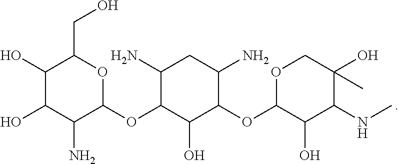 [SMILES: CNC1C(O)C(OC2C(N)CC(N)C(OC3OC(CO)C(O)C(O)C3N)C2O)OCC1(C)O]

The compound may be

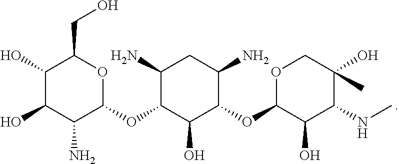 [SMILES: CN[C@@H]1[C@@H](O)[C@@H](O[C@@H]2[C@@H](O)[C@H](O[C@H]3O[C@H](CO)[C@@H](O)[C@H](O)[C@H]3N)[C@@H](N)C[C@H]2N)OC[C@]1(C)O]

The compound may be

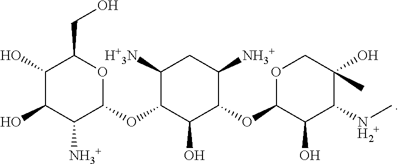

### BRIEF DESCRIPTION OF THE DRAWINGS

FIG. 1 shows the structures of Gentamicins C1, C1a, C2, C2a, C2b, B, B1, A, G418, X2, Sisomicin, Garamine and Ring C, as well as the structure of some of the steroids tested in combination with G418.

FIG. 2 shows the induction of PTC read-through by gentamicin B1 and X2 using the 96-well plate immunofluorescence assay, wherein those not shown on the graph had no read-through activity.

FIG. 3A shows the induction of full-length p53 by gentamicin B1, gentamicin X2, G418 and gentamicin measured by western analysis, where the intensity of the full-length (FL) and truncated p53 (TR) bands is shown relative to the intensity of the truncated p53 band seen in untreated cells and is displayed under the lanes.

FIG. 3B shows the induction of PTC read-through by G418, gentamicin, gentamicin B1 and gentamicin X2 using western analysis, wherein the amount of full-length p53 observed in FIG. 3A was plotted versus the concentration of the different compounds on a log scale.

FIG. 4 shows the induction of full-length p53 by gentamicin G418 in combination with a steroid (A) Dexamethasone; and (B) Betamethasone and Medroxyprogesterone Acetate (Medroxy Pro).

FIG. 5 shows the induction of premature termination codon (PTC) readthrough by gentamicin B1 and gentamicin X2.

FIG. 6 shows induction of PTC readthrough at TGA, TAG and TAA termination codons by gentamicin B1.

FIG. 7 shows induction of PTC readthrough in variety of cancer cell lines—SW900; NCI-H1688; ESS-1; SK-MES-1; HCC1937; H1299; and HCT116.

FIG. 8 shows induction of PTC readthrough in a mouse in vivo assay.

FIG. 9 shows induction of PTC readthrough by Gentamicin B1 in cells derived from patients with rare genetic diseases, wherein Panels A and B show Neuronal Ceroid Lipofuscinosis; Panel C shows Duchenne Muscular Dystrophy; Panel D shows Schimke Immuno-Osseous Dysplasia; and Panel E shows Recessive Dystrophic Epidermolysis Bullosa.

### DETAILED DESCRIPTION

In some embodiments, the compounds described herein may be used to treat or ameliorate various indications, including medical conditions associated with premature termination codons (PTCs) in RNA, including various cancers. The various conditions may be found in TABLE 1.

TABLE 1
Medical Conditions Associated with PTC

| | Gene |
|---|---|
| Medical Condition Associated with PTC | symbol |
| Pk synthase deficiency (p phenotype) | A4GALT |
| Triple-A syndrome | AAAS |
| Ichthyosis, harlequin | ABCA12 |
| Ichthyosiform erythroderma, congenital, nonbullous | ABCA12 |
| Fatal surfactant deficiency | ABCA3 |
| Fundus flavimaculatus, late onset | ABCA4 |
| Stargardt disease | ABCA4 |
| Intrahepatic cholestasis, familial progressive 2 | ABCB11 |
| Intrahepatic cholestasis of pregnancy | ABCB4 |
| Intrahepatic cholestasis, familial progressive | ABCB4 |
| Dubin-Johnson syndrome | ABCC2 |
| Pseudoxanthoma elasticum | ABCC6 |
| Pseudoxanthoma elasticum, autosomal recessive | ABCC6 |
| Pseudoxanthoma elasticum, autosomal dominant | ABCC6 |
| Hyperinsulinism | ABCC8 |
| Hypoglycaemia, persistent hyperinsulinaemic | ABCC8 |
| Adrenoleukodystrophy | ABCD1 |
| Sitosterolaemia | ABCG5 |
| Sitosterolaemia | ABCG8 |
| Chanarin-Dorfman syndrome | ABHD5 |
| Medium chain acyl CoA dehydrogenase deficiency | ACADM |
| Very long chain acyl-CoA dehydrogenase deficiency | ACADVL |
| Alpha actin 3 deficiency | ACTN3 |
| Haemorrhagic telangiectasia 2 | ACVRL1 |
| Adenosine deaminase deficiency | ADA |
| Weill-Marchesani syndrome | ADAMTS10 |
| Thrombotic thrombocytopaenic purpura | ADAMTS13 |
| Upshaw-Schulman syndrome | ADAMTS13 |
| Geleophysic dysplasia | ADAMTSL2 |
| Ectopia lentis, isolated form | ADAMTSL4 |
| Dyschromatosis symmetrica hereditaria | ADAR |
| Parkinson disease, association with | ADH1C |
| Glycogen storage disease 3 | AGL |
| Glycogen storage disease 3a | AGL |
| Renal tubular dysgenesis | AGT |
| Hyperoxaluria | AGXT |
| Molar tooth sign & superior vermian dysplasia | AHI1 |
| Joubert syndrome | AHI1 |
| Pituitary adenoma | AIP |
| Leber congenital amaurosis IV | AIPL1 |
| APECED | AIRE |
| Adenylate kinase deficiency | AK1 |
| Analbuminaemia | ALB |
| Sjoegren-Larsson syndrome | ALDH3A2 |
| Succinic semialdehyde dehydrogenase deficiency | ALDH5A1 |
| Epilepsy, pyridoxine-dependent | ALDH7A1 |
| Aldolase A deficiency | ALDOA |
| Fructose intolerance | ALDOB |
| Alstrom syndrome | ALMS1 |
| Ichthyosis, congenital, autosomal recessive | ALOX12B |
| Ichthyosis, congenital, autosomal recessive | ALOXE3 |
| Hypophosphatasia | ALPL |
| Spastic paralysis, infantile-onset | ALS2 |
| Frontorhiny | ALX3 |
| Amelogenesis imperfecta | AMELX |
| Adenosine monophosphate deaminase deficiency | AMPD1 |
| Spherocytosis | ANK1 |
| Mental retardation | AP1S2 |
| Hermansky-Pudlak syndrome | AP3B1 |
| Adenomatous polyposis coli | APC |
| Apolipoprotein A1 deficiency | APOA1 |
| HDL deficiency with periorbital xanthelasmas | APOA1 |
| HDL deficiency | APOA1 |
| Apolipoprotein A1 deficiency | APOA1 |
| Hypertriglyceridaemia | APOA5 |
| Hypobetalipoproteinaemia | APOB |
| Apolipoprotein B deficiency | APOB |
| Apolipoprotein C2 deficiency | APOC2 |
| Adenine phosphoribosyltransferase deficiency | APRT |
| Diabetes insipidus, nephrogenic | AQP2 |
| Androgen insensitivity syndrome | AR |
| Arginase deficiency | ARG1 |
| X-linked with epilepsy | ARHGEF9 |
| Mental retardation | ARHGEF9 |
| Bardet-Biedl syndrome | ARL6 |
| Cancer, association with | ARL11 |
| Metachromatic leukodystrophy | ARSA |
| Mucopolysaccharidosis VI | ARSB |
| Chondrodysplasia punctata | ARSE |
| Dombrock blood group variation | ART4 |
| Lissencephaly, X-linked, with abnormal genitalia | ARX |
| Argininosuccinate lyase deficiency | ASL |
| Canavan disease | ASPA |
| Primary microcephaly | ASPM |
| Polycystic kidney disease 1 | ASS1 |
| Citrullinaemia | ASS1 |
| Ataxia telangiectasia | ATM |
| Mantle cell lymphoma | ATM |
| Hemiplegic migraine | ATP1A2 |
| Darier disease | ATP2A2 |
| Hailey-Hailey disease | ATP2C1 |
| Cutis laxa, autosomal recessive, type 2 | ATP6V0A2 |
| Distal renal tubular acidosis, autosomal recessive | ATP6V0A4 |
| Menkes syndrome | ATP7A |
| Wilson disease | ATP7B |
| Intrahepatic cholestasis, familial progressive | ATP8B1 |
| Intrahepatic cholestasis, benign recurrent | ATP8B1 |
| ATRX syndrome | ATRX |
| 3-methylglutaconic aciduria type 1 | AUH |
| Diabetes insipidus, neurohypophyseal | AVP |
| Diabetes insipidus, central | AVP |
| Diabetes insipidus, nephrogenic | AVPR2 |
| Tooth agenesis and colorectal cancer | AXIN2 |
| B3GALNT1 deficiency (P2K phenotype) | B3GALNT1 |
| Cholinesterasaemia | BCHE |
| Butyrylcholinesterase variant | BCHE |
| Maple syrup urine disease | BCKDHA |
| Maple syrup urine disease | BCKDHB |
| Oculofaciocardiodental syndrome | BCOR |
| Bestrophinopathy | BEST1 |
| Cleft lip and palate | BMP4 |
| Juvenile polyposis syndrome | BMPR1A |
| Polyposis, juvenile intestinal | BMPR1A |
| Pulmonary hypertension, primary | BMPR2 |
| Pulmonary arterial hypertension | BMPR2 |
| Pulmonary hypertension, primary | BMPR2 |
| Breast cancer | BRCA1 |
| Breast and/or ovarian cancer | BRCA1 |
| Breast cancer | BRCA2 |
| Breast and/or ovarian cancer | BRCA2 |
| Berardinelli-Seip lipodystrophy | BSCL2 |
| Bartter syndrome with sensorineural deafness | BSND |
| Biotinidase deficiency | BTD |
| Agammaglobulinaemia | BTK |
| Premature chromatid separation syndrome | BUB1B |
| Complement C1S deficiency | C1S |
| Complement C3 deficiency | C3 |
| Complement C5 deficiency | C5 |
| Complement C7 deficiency | C7 |
| Complement C8 alpha-gamma deficiency | C8A |
| Carbonic anhydrase deficiency | CA2 |
| Cone-rod synaptic disorder | CABP4 |
| Episodic ataxia 2 | CACNA1A |
| Night blindness, congenital stationary, incomplete | CACNA1F |
| Muscular dystrophy, limb girdle | CAPN3 |
| Ventricular tachycardia, polymorphic | CASQ2 |
| Hypercalcaemia, hypocalciuric | CASR |
| Berardinelli-Seip lipodystrophy | CAV1 |
| Joubert syndrome | CC2D2A |
| Joubert syndrome | CC2D2A |
| Cerebral cavernous malformations | CCM2 |
| CD36 deficiency | CD36 |
| Hyper-IgM syndrome | CD40LG |
| Cromer blood group | CD55 |
| Agammaglobulinaemia | CD79B |
| Hyperparathyroidism, primary | CDC73 |
| Gastric cancer | CDH1 |
| Usher syndrome 1d | CDH23 |
| Hypotrichosis with juvenile macular dystrophy | CDH3 |
| Rett syndrome, atypical | CDKL5 |
| Pituitary and parathyroid tumours | CDKN1B |
| Melanoma | CDKN2A |
| Hypotrichosis simplex of the scalp | CDSN |
| Bardet-Biedl syndrome | CEP290 |
| Leber congenital amaurosis | CEP290 |
| Cholesterol ester transfer protein deficiency | CETP |
| Drusen, basal laminar | CFH |
| Cystic fibrosis | CFTR |
| Congenital absence of vas deferens | CFTR |
| Elevated sweat chloride concentration | CFTR |
| CHARGE syndrome | CHD7 |
| Choroideraemia | CHM |
| Frontotemporal dementia | CHMP2B |
| Fetal akinesia deformation sequence disorder | CHRND |
| Congenital myasthenic syndrome | CHRNE |
| Slow channel myasthenic syndrome | CHRNE |
| Macular corneal dystrophy, type 1 | CHST6 |
| Immunodeficiency | CIITA |
| Myotonia congenita | CLCN1 |
| Myotonia, Becker | CLCN1 |
| Myotonia | CLCN1 |
| Low molecular weight proteinuria | CLCN5 |
| Dent disease | CLCN5 |
| Dent (Japan) disease | CLCN5 |
| Bartter syndrome 4, digenic | CLCNKA |
| Bartter syndrome 3 | CLCNKB |
| Neuronal ceroid lipofuscinosis, juvenile | CLN3 |
| Neuronal ceroid lipofuscinosis, late infantile | CLN5 |
| Neuronal ceroid lipofuscinosis, late infantile | CLN6 |
| Retinitis pigmentosa | CNGA1 |
| Achromatopsia | CNGB3 |
| Congenital disorder of glycosylation IIh | COG8 |
| Metaphyseal chondrodysplasia, Schmid | COL10A1 |
| Stickler syndrome, without eye involvement | COL11A2 |
| Epidermolysis bullosa | COL17A1 |
| Epidermolysis bullosa, junctional | COL17A1 |
| Epidermolysis bullosa, atrophic benign | COL17A1 |
| Osteogenesis imperfecta I | COL1A1 |
| Osteogenesis imperfecta | COL1A1 |
| Ehlers-Danlos syndrome VII | COL1A2 |
| Stickler syndrome | COL2A1 |
| Spondyloperipheral dysplasia | COL2A1 |
| Ehlers-Danlos syndrome IV | COL3A1 |
| Alport syndrome | COL4A3 |
| Alport syndrome | COL4A5 |
| Ullrich congenital muscular dystrophy | COL6A1 |
| Myosclerosis myopathy | COL6A2 |
| Ullrich congenital muscular dystrophy | COL6A3 |
| Epidermolysis bullosa | COL7A1 |
| Epidermolysis bullosa dystrophica | COL7A1 |
| Endplate acetylcholinesterase deficiency | COLQ |
| Aceruloplasminaemia with diabetes | CP |
| Aceruloplasminaemia | CP |
| Coproporphyria | CPOX |
| Harderoporphyria | CPOX |
| Coproporphyria | CPOX |
| Carbamoyl phosphate synthetase I deficiency | CPS1 |
| Carnitine palmitoyltransferase 1 deficiency | CPT1A |
| Leber congenital amaurosis | CRB1 |
| Mental retardation, non-syndromic, autosomal recessive | CRBN |
| Rubinstein-Taybi syndrome | CREBBP |
| Crisponi syndrome | CRLF1 |
| Congenital cataract | CRYAA |
| Cataract, autosomal dominant | CRYBB1 |
| Cataract | CRYGC |
| Cataract, pediatric | CRYGD |
| Cataract | CRYGD |
| Cystinosis | CTNS |
| Pancreatitis, chronic | CTRC |
| Papillon-Lefevre syndrome | CTSC |
| Pycnodysostosis | CTSK |
| 3-M syndrome | CUL7 |
| Methaemoglobinaemia 2 | CYB5R3 |
| Methaemoglobinaemia | CYB5R3 |
| Chronic granulomatous disease | CYBA |
| Chronic granulomatous disease | CYBB |
| Trichoepithelioma, multiple familial | CYLD |
| Adrenal hyperplasia | CYP11B1 |
| Steroid-11 beta-hydroxylase deficiency | CYP11B1 |
| Adrenal hyperplasia | CYP11B1 |
| Steroid-11 beta-hydroxylase deficiency | CYP11B1 |
| 17-alpha-hydroxylase/17,20-lyase deficiency | CYP17A1 |
| Glaucoma, primary congenital | CYP1B1 |
| Adrenal hyperplasia | CYP21A2 |
| Non-classic 21-hydroxylase deficiency | CYP21A2 |
| Adrenal hyperplasia | CYP21A2 |
| Pseudovitamin D-deficiency rickets | CYP27B1 |
| Null allele | CYP2A13 |
| Cytochrome P450 deficiency | CYP2D6 |
| CYP2G deficiency, association with | CYP2G2P |
| Null allele | CYP4A22 |
| Bietti crystalline corneoretinal dystrophy | CYP4V2 |
| Spastic paraplegia | CYP7B1 |
| Maple syrup urine disease | DBT |
| Immunodeficiency, severe combined | DCLRE1C |
| Subcortical band heterotopia | DCX |
| Double cortex syndrome | DCX |
| Usher syndrome 2 | DFNB31 |
| Progressive hearing loss, autosomal recessive | DFNB59 |
| Mitochondrial DNA depletion syndrome | DGUOK |
| Smith-Lemli-Opitz syndrome | DHCR7 |
| Spondylocostal dysostosis | DLL3 |
| Muscular dystrophy, Duchenne | DMD |
| Dystrophinopathy | DMD |
| Muscular dystrophy, Becker | DMD |
| Primary ciliary dyskinesia and situs inversus | DNAH11 |
| Primary ciliary dyskinesia | DNAH5 |
| Primary ciliary dyskinesia | DNAI1 |
| Primary ciliary dyskinesia | DNAI2 |
| Systemic lupus erythematosus | DNASE1 |
| Immunodeficiency, centromeric instability and facial anomalies | DNMT3B |
| syndrome | |
| Dihydropyrimidine dehydrogenase deficiency | DPYD |
| Receptor deficiency | DRD5 |
| Striate palmoplantar keratoderma | DSG1 |
| Cardiomyopathy, arrhythmogenic right ventricular | DSG2 |
| Dilated cardiomyopathy, woolly hair, keratoderma | DSP |
| Dentinogenesis imperfecta Shields type II | DSPP |
| Hypothyroidism | DUOX2 |
| Hypothyroidism, transient | DUOX2 |
| Hypothyroidism, transient | DUOX2 |
| Hypothyroidism | DUOX2 |
| Hypothyroidism | DUOXA2 |
| Smith-McCort dysplasia | DYM |
| Dyggve-Melchior-Clausen syndrome | DYM |
| Muscular dystrophy, limb girdle | DYSF |
| Miyoshi myopathy | DYSF |
| Chondrodysplasia punctata, X-linked | EBP |
| CHILD syndrome | EBP |
| Lipoid proteinosis | ECM1 |
| Ectodermal dysplasia | EDA |
| Ectodermal dysplasia, hypohidrotic | EDAR |
| Waardenburg-Hirschsprung disease | EDNRB |
| ABCD syndrome | EDNRB |
| Craniofrontonasal syndrome | EFNB1 |
| Erythrocytosis | EGLN1 |
| Mental retardation | EHMT1 |
| Wolcott-Rallison syndrome | EIF2AK3 |
| Leukoencephalopathy with vanishing white matter | EIF2B4 |
| Prostate cancer | ELAC2 |
| Supravalvular aortic stenosis | ELN |
| Amelogenesis imperfecta, hypoplastic | ENAM |
| Haemorrhagic telangiectasia 1 | ENG |
| Idiopathic infantile arterial calcification | ENPP1 |
| Prostate cancer, increased risk, in African Americans, association with | EPHB2 |
| Erythrocytosis | EPOR |
| Xeroderma pigmentosum (B) | ERCC3 |
| Xeroderma pigmentosum/Cockayne syndrome | ERCC3 |
| Cockayne syndrome | ERCC8 |
| SC Phocomelia | ESCO2 |
| Glutaricacidaemia 2a | ETFA |
| Electron transfer flavoprotein deficiency | ETFA |
| Multiple exostoses | EXT1 |
| Multiple exostoses | EXT2 |
| Branchio-oto-renal/branchiootic syndrome | EYA1 |
| Branchio-oto-renal syndrome | EYA1 |
| Factor XI deficiency | F11 |
| Factor XIII deficiency | F13A1 |
| Factor V deficiency | F5 |
| Factor VII deficiency | F7 |
| Haemophilia A | F8 |
| Haemophilia B | F9 |
| Tyrosinaemia 1 | FAH |
| Amelogenesis imperfecta, hypoplastic local | FAM83H |
| Amelogenesis imperfecta, hypocalcified | FAM83H |
| Fanconi anaemia | FANCA |
| Fanconi anaemia | FANCC |
| Fanconi anaemia | FANCG |
| Cytochrome c oxidase deficiency | FASTKD2 |
| Marfan syndrome | FBN1 |
| Ectopia lentis | FBN1 |
| Fibrillinopathy | FBN1 |
| Kindler syndrome | FERMT1 |
| Afibrinogenaemia | FGA |
| Dysfibrinogenaemia | FGA |
| Hypofibrinogenaemia | FGB |
| Afibrinogenaemia | FGB |
| Charcot-Marie-Tooth disease 4H | FGD4 |
| Lacrimo-auriculo-dento-digital syndrome | FGF10 |
| Kallmann syndrome | FGFR1 |
| Afibrinogenaemia | FGG |
| Leiomyomatosis and renal cell cancer | FH |
| Fumarase deficiency | FH |
| Cutaneous leiomyomatosis | FH |
| Muscular dystrophy, Fukuyama | FKTN |
| Pneumothorax, primary spontaneous | FLCN |
| Birt-Hogg-Dub syndrome | FLCN |
| Ichthyosis vulgaris | FLG |
| Ichthyosis vulgaris | flg10.2 |
| Heterotopia, periventricular | FLNA |
| Myopathy, myofibrillar | FLNC |
| FMO1 variant | FMO1 |
| FMO2 variant | FMO2 |
| Trimethylaminuria | FMO3 |
| fmo6 variant | FMO6P |
| Axenfeld-Rieger & Peters' anomaly | FOXC1 |
| Axenfeld-Rieger anomaly | FOXC1 |
| Lymphoedema-distichiasis | FOXC2 |
| Aphakia, congenital, primary | FOXE3 |
| ACD/MPV with cardiovascular malformations | FOXF1 |
| Blepharophimosis/ptosis/epicanthus inversus syndrome | FOXL2 |
| Developmental verbal dyspraxia | FOXP2 |
| Follicle-stimulating hormone deficiency | FSHB |
| Mental retardation | FTSJ1 |
| Fucosidosis | FUCA1 |
| H antigen, Bombay phenotype | FUT1 |
| H antigen, para-Bombay phenotype | FUT1 |
| Non-secretor phenotype | FUT2 |
| Fucosyltransferase deficiency | FUT2 |
| Fucosyltransferase deficiency | FUT6 |
| Friedreich ataxia | FXN |
| Exudative vitreoretinopathy | FZD4 |
| Glycogen storage disease 1a | G6PC |
| Glucose-6-phosphate dehydrogenase deficiency | G6PD |
| Glycogen storage disease 2 | GAA |
| Krabbe disease | GALC |
| Galactosaemia epimerase deficiency | GALE |
| Mucopolysaccharidosis IVa | GALNS |
| Tumoural calcinosis | GALNT3 |
| Galactosaemia | GALT |
| Giant axonal neuropathy | GAN |
| Hypoparathyroidism, deafness and renal dysplasia | GATA3 |
| Gaucher disease 2 | GBA |
| Glycogen storage disease 4 | GBE1 |
| Dystonia, dopa-responsive | GCH1 |
| Diabetes, NIDDM | GCK |
| Diabetes, MODY2 | GCK |
| Diabetes, MODY | GCK |
| Congenital cataract | GCNT2 |
| Demyelinating peripheral neuropathy | GDAP1 |
| Charcot-Marie-Tooth disease 4A | GDAP1 |
| Charcot-Marie-Tooth disease, autosomal recessive | GDAP1 |
| Brachydactyly, type C | GDF5 |
| Laron dwarfism | GHR |
| Growth hormone insensitivity | GHR |
| Growth hormone deficiency | GHRHR |
| Growth hormone deficiency, isolated | GHSR |
| Oculodentodigital dysplasia | GJA1 |
| Charcot-Marie-Tooth disease | GJB1 |
| Deafness, autosomal recessive 1 | GJB2 |
| Deafness | GJB2 |
| Deafness, non-syndromic, autosomal dominant | GJB3 |
| Pelizaeus-Merzbacher-like disease | GJC2 |
| Glycerol kinase deficiency | GK |
| Fabry disease | GLA |
| Gangliosidosis GM1 | GLB1 |
| Hyperglycinaemia, non-ketotic | GLDC |
| Hyperglycinaemia, non-ketotic | GLDC |
| Hyperglycinaemia, non-ketotic | GLDC |
| Hyperglycinaemia, non-ketotic | GLDC |
| Pallister-Hall syndrome | GLI3 |
| Greig cephalopolysyndactyly syndrome | GLI3 |
| Postaxial polydactyly A/B | GLI3 |
| Hyperekplexia | GLRA1 |
| Gangliosidosis GM2 | GM2A |
| Albright hereditary osteodystrophy | GNAS |
| Progressive osseous heteroplasia | GNAS |
| Mucolipidosis II | GNPTAB |
| Mucopolysaccharidosis IIId | GNS |
| Bernard-Soulier syndrome | GP1BA |
| Giant platelet disorder | GP1BB |
| Bernard-Soulier syndrome | GP9 |
| Simpson-Golabi-Behmel syndrome | GPC3 |
| Glucosephosphate isomerase deficiency | GPI |
| Albinism, ocular | GPR143 |
| Febrile and afebrile seizures | GPR98 |
| Hyperoxaluria II | GRHPR |
| Frontotemporal dementia | GRN |
| Alzheimer disease | GRN |
| Glutathione synthetase deficiency | GSS |
| Leber congenital amaurosis | GUCY2D |
| Mucopolysaccharidosis VII | GUSB |
| Hypoglycaemia, hyperinsulinaemic | HADH |
| Thalassaemia alpha | HBA2 |
| Thalassaemia beta | HBB |
| Microphthalmia, syndromic 7 | HCCS |
| Tay-Sachs disease | HEXA |
| Sandhoff disease | HEXB |
| Haemochromatosis | HFE |
| Haemochromatosis | HFE2 |
| Alkaptonuria | HGD |
| Mucopolysaccharidosis IIIC | HGSNAT |
| HLA-A null allele | HLA-A |
| HLA-B null allele | HLA-B |
| Holocarboxylase synthetase deficiency | HLCS |
| Porphyria, acute intermittent | HMBS |
| HMG-CoA lyase deficiency | HMGCL |
| 3-hydroxy-3-methylglutaric aciduria | HMGCL |
| HMG-CoA lyase deficiency | HMGCL |
| Diabetes, MODY3 | HNF1A |
| Diabetes, MODY | HNF1B |
| GCKD with early-onset diabetes | HNF1B |
| Diabetes, MODY1 | HNF4A |
| Hand-foot-genital syndrome | HOXA13 |
| Tyrosinaemia 3 | HPD |
| Lesch-Nyhan syndrome | HPRT1 |
| Hypoxanthine guanine phosphoribosyltransferase deficiency | HPRT1 |
| Hermansky-Pudlak syndrome | HPS1 |
| Hermansky-Pudlak syndrome | HPS4 |
| Atrichia with papular lesions | HR |
| Congenital atrichia | HR |
| Adrenal hyperplasia | HSD3B2 |
| Cataract, autosomal recessive | hsf4b |
| Schwartz-Jampel syndrome type 1 | HSPG2 |
| CARASIL | HTRA1 |
| Mucopolysaccharidosis II | IDS |
| Scheie syndrome | IDUA |
| Hurler syndrome | IDUA |
| Reduced activity | IFIH1 |
| Growth retardation | IGF1R |
| Acid-labile subunit deficiency | IGFALS |
| Spinal muscular atrophy with respiratory distress 1 | IGHMBP2 |
| Spinal muscular atrophy with respiratory distress 1 | IGHMBP2 |
| Spinal muscular atrophy with respiratory distress 1 | IGHMBP2 |
| Incontinentia pigmenti | IKBKG |
| Incontinentia pigmenti, familial | IKBKG |
| Mental retardation, X-linked | IL1RAPL1 |
| Immunodeficiency, severe combined | IL2RG |
| Immunodeficiency, severe combined | IL7R |
| Leprechaunism | INSR |
| Insulin resistance | INSR |
| Insulin resistance A | INSR |
| Senior-Loken syndrome 5 | IQCB1 |
| Van der Woude syndrome | IRF6 |
| Popliteal pterygium syndrome | IRF6 |
| Diabetes, type 2 | ISL1 |
| Glanzmann thrombasthenia | ITGA2B |
| Leukocyte adhesion deficiency | ITGB2 |
| Glanzmann thrombasthenia | ITGB3 |
| Epidermolysis bullosa with pyloric atresia | ITGB4 |
| Alagille syndrome | JAG1 |
| Immunodeficiency, severe combined | JAK3 |
| Kallmann syndrome | KAL1 |
| Atrial fibrillation | KCNA5 |
| Miscarriage and intrauterine foetal loss | KCNH2 |
| Long QT syndrome | KCNH2 |
| Hyperinsulinism | KCNJ11 |
| Long QT syndrome | KCNQ1 |
| Cone dystrophy with supernormal rod ERG | KCNV2 |
| Mental retardation, X-linked | KDM5C |
| Kell blood group variation | KEL |
| K(null) phenotype | KEL |
| Cornea plana 2 | KERA |
| Goldberg-Shprintzen syndrome | KIAA1279 |
| Piebaldism | KIT |
| Prostate cancer | KLF6 |
| Cerebral cavernous malformations | KRIT1 |
| Dowling-Degos disease | KRT5 |
| Epidermolysis bullosa, Dowling-Meara | KRT5 |
| Epidermolysis bullosa simplex | KRT5 |
| Epidermolytic hyperkeratosis | KRT10 |
| Epidermolysis bullosa, Koebner | KRT14 |
| Naegeli syndrome | KRT14 |
| Dermatopathia pigmentosa reticularis | KRT14 |
| Hydrocephalus, X-linked | L1CAM |
| L-2-Hydroxyglutaric aciduria | L2HGDH |
| Muscular dystrophy, merosin deficient | LAMA2 |
| Laminin alpha 2 chain deficiency, partial | LAMA2 |
| Epidermolysis bullosa, Herlitz | LAMA3 |
| Laryngo-onycho-cutaneous syndrome | LAMA3 |
| Cardiomyopathy, dilated | LAMA4 |
| Epidermolysis bullosa, Herlitz | LAMB3 |
| Epidermolysis bullosa, junctional | LAMB3 |
| Epidermolysis bullosa, Herlitz | LAMC2 |
| Epidermolysis bullosa, junctional | LAMC2 |
| Danon disease | LAMP2 |
| Pelger-Huet anomaly | LBR |
| Leber congenital amaurosis | LCA5 |
| Lecithin:cholesterol acyltransferase deficiency | LCAT |
| Lactase deficiency, congenital | LCT |
| Lactate dehydrogenase deficiency | LDHB |
| Hypercholesterolaemia | LDLR |
| Left-right axis malformation | LEFTY2 |
| Osteopoikilosis | LEMD3 |
| Leydig cell hypoplasia | LHCGR |
| Pseudohermaphroditism | LHCGR |
| Wolman syndrome | LIPA |
| Factor V and factor VIII deficiency, combined | LMAN1 |
| Factor V and factor VIII deficiency, combined | LMAN1 |
| Muscular dystrophy, limb girdle | LMNA |
| Muscular dystrophy, Emery-Dreifuss | LMNA |
| Cardiomyopathy, dilated | LMNA |
| Nail patella syndrome | LMX1B |
| Lipoprotein lipase deficiency | LPL |
| Hypertriglyceridaemia | LPL |
| Lipoprotein lipase deficiency, association with | LPL |
| Deafness, non-syndromic | lrtomt2 |
| Oligodontia | LTBP3 |
| Chediak-Higashi syndrome | LYST |
| Hypospadias | MAMLD1 |
| Mannosidosis, alpha | MAN2B1 |
| Mannosidosis, beta, lysosomal | MANBA |
| Obesity, autosomal dominant | MC4R |
| 3-methylcrotonyl-CoA carboxylase deficiency | MCCC1 |
| 3-methylcrotonyl-CoA carboxylase deficiency | MCCC2 |
| Methylmalonic aciduria | MCEE |
| Factor V and Factor VIII deficiency, combined | MCFD2 |
| Mucolipidosis IV | MCOLN1 |
| Rett syndrome | MECP2 |
| Myocardial infarction | MEF2A |
| Mediterranean fever, familial | MEFV |
| Multiple endocrine neoplasia 1 | MEN1 |
| Spondylocostal dysostosis | MESP2 |
| Neuronal ceroid lipofuscinoses, late infantile | MFSD8 |
| Opitz G/BBB syndrome | MID1 |
| Bardet-Biedl syndrome | MKKS |
| Colorectal cancer, non-polyposis | MLH1 |
| Colorectal cancer, young-onset | MLH1 |
| Colorectal cancer | MLH1 |
| Gastrointestinal cancer | MLH1 |
| Lynch syndrome-associated breast cancer | MLH1 |
| Colorectal cancer, early onset | MLH1 |
| Methylmalonic aciduria | MMAB |
| Methylmalonic aciduria, cblB type | MMAB |
| Fetomaternal alloimmunisation | MME |
| Osteolysis, idiopathic, Saudi type | MMP2 |
| Currarino syndrome | MNX1 |
| Xanthinuria, type 2 | MOCOS |
| Amegakaryocytic thrombocytopaenia, congenital | MPL |
| Mercaptopyruvate sulphurtransferase deficiency, association with | MPST |
| Mitochondrial DNA depletion syndrome, hepatocerebral | MPV17 |
| Charcot-Marie-Tooth disease 1b | MPZ |
| Charcot-Marie-Tooth disease 1 | MPZ |
| Ataxia telangiectasia-like disease | MRE11A |
| Mitochondrial respiratory chain disorder | MRPS16 |
| Atopy | MS4A2 |
| Colorectal cancer, non-polyposis | MSH2 |
| Colorectal cancer, non-polyposis | MSH6 |
| Prostate cancer | MSR1 |
| Witkop syndrome | MSX1 |
| Homocystinuria | MTHFR |
| Methylenetetrahydrofolate reductase deficiency | MTHFR |
| Homocystinuria | MTHFR |
| Myotubular myopathy | MTM1 |
| Methionine synthase deficiency | MTR |
| Methionine synthase reductase deficiency | MTRR |
| Abetalipoproteinaemia | MTTP |
| Methylmalonic aciduria | MUT |
| Mevalonic kinase deficiency | MVK |
| Hyperimmunoglobulin D and periodic fever syndrome | MVK |
| Hyperimmunoglobulin D and periodic fever syndrome | MVK |
| Cardiomyopathy, hypertrophic | MYBPC3 |
| Cardiomyopathy, hypertrophic | MYBPC3 |
| Feingold syndrome | MYCN |
| Hearing impairment | MYH14 |
| Cardiomyopathy, hypertrophic | MYH7 |
| May-Hegglin anomaly | MYH9 |
| Deafness, non-syndromic, autosomal recessive | MYO15A |
| Sensorineural deafness, nonsyndromic | MYO1A |
| Microvillus inclusion disease | MYO5B |
| Deafness, autosomal dominant 22 | MYO6 |
| Deafness, autosomal recessive | MYO6 |
| Usher syndrome 1b | MYO7A |
| Sanfilippo syndrome B | NAGLU |
| Fertility defects | NBN |
| Chronic granulomatous disease | NCF1 |
| Chronic granulomatous disease | NCF2 |
| Norrie disease | NDP |
| Mitochondrial complex I deficiency | NDUFAF2 |
| Complex 1 deficiency | NDUFS4 |
| Nemaline myopathy | NEB |
| Charcot-Marie-Tooth disease | NEFL |
| Sialidosis | NEU1 |
| Sialidosis 2 | NEU1 |
| Neurofibromatosis 1 | NF1 |
| Neurofibromatosis 2 | NF2 |
| Ectodermal dysplasia, anhidrotic with immune deficiency | NFKBIA |
| Myoclonic epilepsy of Lafora | NHLRC1 |
| Ichthyosis, autosomal recessive | NIPAL4 |
| Cornelia de Lange syndrome | NIPBL |
| Benign hereditary chorea | NKX2-1 |
| Hypothyroidism | NKX2-1 |
| Periodic fever syndrome | NLRP12 |
| Familial cold autoinflammatory syndrome | NLRP3 |
| Primary ciliary dyskinesia | NME8 |
| Stapes ankylosis with broad thumb and toes | NOG |
| Niemann-Pick disease C | NPC1 |
| Niemann-Pick type C2 disease | NPC2 |
| Nephronophthisis 1 | NPHP1 |
| Nephronophthisis 3 | NPHP3 |
| Nephronophthisis 4 | NPHP4 |
| Congenital nephrotic syndrome, Finnish type | NPHS1 |
| Nephrotic syndrome | NPHS1 |
| Nephrotic syndrome, steroid resistant | NPHS2 |
| Nephrotic syndrome | NPHS2 |
| Acromesomelic dysplasia, Maroteaux type | NPR2 |
| Adrenal hypoplasia | NR0B1 |
| Enhanced S cone syndrome | NR2E3 |
| Pseudohypoaldosteronism 1 | NR3C2 |
| XY sex reversal, without adrenal failure | NR5A1 |
| Sotos syndrome | NSD1 |
| CHILD syndrome | NSDHL |
| Pain insensitivity, congenital | NTRK1 |
| Gyrate atrophy | OAT |
| Albinism, oculocutaneous II | OCA2 |
| Lowe oculocerebrorenal syndrome | OCRL |
| Oral-facial-digital syndrome 1 | OFD1 |
| Optic atrophy 1 | OPA1 |
| Mental retardation syndrome, X-linked | OPHN1 |
| X-linked cone dystrophy | orf15 |
| Atrophic macular degeneration | orf15 |
| Retinitis pigmentosa, X-linked | orf15 |
| Osteopetrosis, autosomal recessive | OSTM1 |
| Ornithine transcarbamylase deficiency | OTC |
| Ornithine transcarbamylase deficiency | OTC |
| Ornithine transcarbamylase deficiency | OTC |
| Deafness, autosomal recessive 9 | OTOF |
| Deafness, non-syndromic | OTOF |
| Lissencephaly, isolated | PAFAH1B1 |
| Subcortical band heterotopia | PAFAH1B1 |
| Phenylketonuria | PAH |
| HARP syndrome | PANK2 |
| Pantothenate kinase-associated neurodegeneration | PANK2 |
| Spondyloepiphyseal dysplasia | PAPSS2 |
| Parkinsonism, juvenile, autosomal recessive | PARK2 |
| Renal hypoplasia | PAX2 |
| Waardenburg syndrome | PAX3 |
| Aniridia | PAX6 |
| Oligodontia | PAX9 |
| Hyperphenylalaninaemia | PCBD1 |
| Propionic acidaemia | PCCA |
| Propionic acidaemia | PCCB |
| Usher syndrome 1f | PCDH15 |
| Epilepsy and mental retardation limited to females | PCDH19 |
| Schizophrenia | PCM1 |
| Obesity and impaired prohormone processing | PCSK1 |
| Low LDL cholesterol | PCSK9 |
| Cerebral cavernous malformation | PDCD10 |
| Retinitis pigmentosa | PDE6B |
| Pyruvate dehydrogenase deficiency | PDHA1 |
| Pyruvate dehydrogenase complex deficiency | PDHX |
| Pyruvate dehydrogenase phosphatase deficiency | PDP1 |
| Prolidase deficiency | PEPD |
| Zellweger syndrome | PEX1 |
| Peroxisome biogenesis disorder | PEX1 |
| Neonatal adrenoleukodystrophy | PEX10 |
| Zellweger syndrome H | PEX13 |
| Zellweger syndrome | PEX14 |
| Zellweger syndrome, complementation group D | PEX16 |
| Rhizomelic chondrodysplasia punctata | PEX7 |
| Glycogen storage disease 7 | PFKM |
| Rickets, hypophosphataemic | PHEX |
| X-linked mental retardation & cleft lip/palate | PHF8 |
| Phosphorylase kinase deficiency | PHKA1 |
| Liver glycogenosis 1 | PHKA2 |
| Liver glycogenosis | PHKB |
| Fibrosis of extraocular muscles type 2 | PHOX2A |
| Central hypoventilation syndrome | PHOX2B |
| Parkinson disease, early-onset | PINK1 |
| Axenfeld-Rieger syndrome | PITX2 |
| Polycystic kidney disease 1 | PKD1 |
| Polycystic kidney disease 2 | PKD2 |
| Polycystic kidney disease | PKHD1 |
| Pyruvate kinase deficiency | PKLR |
| Haemolytic anaemia | PKLR |
| Pyruvate kinase deficiency | PKLR |
| Ectodermal dysplasia/skin fragility syndrome | PKP1 |
| Infantile neuroaxonal dystrophy 1 | PLA2G6 |
| Epidermolysis bullosa with pyloric atresia | PLEC |
| Muscular dystrophy with epidermolysis bullosa | PLEC |
| Epidermolysis bullosa simplex | PLEC |
| Plasminogen deficiency | PLG |
| Ehlers-Danlos syndrome VI | PLOD1 |
| Pelizaeus-Merzbacher disease | PLP1 |
| Spastic paraplegia | PLP1 |
| Congenital disorder of glycosylation 1a | PMM2 |
| Turcot syndrome | PMS2 |
| PNPO deficiency | PNPO |
| Alpers syndrome | POLG |
| Xeroderma pigmentosum, variant | POLH |
| Xeroderma pigmentosum, variant | POLH |
| Obesity | POMC |
| Walker-Warburg syndrome | POMT1 |
| Focal dermal hypoplasia | PORCN |
| Pituitary hormone deficiency | POU1F1 |
| Partial lipodystrophy | PPARG |
| Porphyria, variegate | PPOX |
| Neuronal ceroid lipofuscinosis, juvenile | PPT1 |
| Neuronal ceroid lipofuscinosis, infantile | PPT1 |
| Neuronal ceroid lipofuscinosis, late infantile | PPT1 |
| Haemophagocytic lymphohistiocytosis, familial | PRF1 |
| Perforin deficiency | PRF1 |
| Camptodactyly-arthropathy-coxa vara-pericarditis | PRG4 |
| Carney complex | PRKAR1A |
| Azoospermia | PRM2 |
| Protein C deficiency | PROC |
| Hypogonadotropic hypogonadism | PROKR2 |
| Hypogonadotropic hypogonadism | PROP1 |
| Protein S deficiency | PROS1 |
| High myopia | PRPH |
| Pattern dystrophy | PRPH2 |
| Pancreatitis, protection against | PRSS1 |
| Dejerine-Sottas syndrome | PRX |
| Charcot-Marie-Tooth disease 4 | PRX |
| Gaucher disease, atypical | PSAP |
| Nevoid basal cell carcinoma syndrome | PTCH1 |
| Cowden disease | PTEN |
| Hypertension | PTGIS |
| Osteochondrodysplasia, Blomstrand, type 1 | PTH1R |
| Mitochondrial myopathy and sideroblastic anaemia | PUS1 |
| McArdle disease | PYGM |
| Dihydropteridine reductase deficiency | QDPR |
| Acrocephalopolysyndactyly, type II | RAB23 |
| Immunodeficiency, severe combined | RAG2 |
| Immunodeficiency, severe combined, B cell −ve | RAG2 |
| Omenn syndrome | RAG2 |
| Smith-Magenis syndrome | RAI1 |
| Anophthalmia | RAX |
| Retinoblastoma | RB1 |
| RAPADILINO syndrome | RECQL4 |
| Spastic paraplegia 31 | REEP1 |
| Hirschsprung disease | RET |
| MHC class II deficiency | RFXANK |
| Retinitis pigmentosa | RHO |
| Ribonuclease L deficiency | RNASEL |
| Brachydactyly, type B | ROR2 |
| Robinow syndrome, autosomal recessive | ROR2 |
| Brachydactyly, type B | ROR2 |
| Retinitis pigmentosa | RP1 |
| Retinitis pigmentosa, X-linked | RP2 |
| Leber congenital amaurosis | RPE65 |
| Retinitis pigmentosa, X-linked | RPGR |
| Diamond-Blackfan anaemia | RPS24 |
| Coffin-Lowry syndrome | RPS6KA3 |
| Mitochondrial DNA depletion syndrome | RRM2B |
| Retinoschisis, X linked juvenile | RS1 |
| Platelet disorder, familial | RUNX1 |
| Cleidocranial dysplasia | RUNX2 |
| Townes-Brocks syndrome | SALL1 |
| Goldenhar syndrome | SALL1 |
| Townes-Brocks syndrome | SALL1 |
| Okihiro syndrome | SALL4 |
| Tumoural calcinosis, normophosphataemic | SAMD9 |
| Chylomicron retention disease | SAR1B |
| Cleft palate, osteoporosis and cognitive defects | SATB2 |
| Charcot-Marie-Tooth disease 4b2 | SBF2 |
| Action myoclonus-renal failure syndrome | SCARB2 |
| Myoclonic epilepsy of infancy | SCN1A |
| Dravet syndrome or Dravet syndrome C or Dravet syndrome B | SCN1A |
| Generalized epilepsy with febrile seizures plus | SCN1A |
| Intractable epilepsy | SCN1A |
| Intractable epilepsy and mental decline | SCN2A |
| Brugada syndrome | SCN5A |
| Cardiac conduction disease | SCN5A |
| Channelopathy-associated insensitivity to pain | SCN9A |
| Cardioencephalomyopathy, fatal infantile | SCO2 |
| Cytochrome c oxidase deficiency | SCO2 |
| Leigh syndrome | SDHA |
| Phaeochromocytoma | SDHB |
| Paraganglioma, autosomal dominant 3 | SDHC |
| Paraganglioma | SDHD |
| SEPN-related myopathy | SEPN1 |
| Antitrypsin alpha 1 deficiency | SERPINA1 |
| Venous thromboembolic disease | SERPINA10 |
| Thyroxine-binding globulin deficiency | SERPINA7 |
| Antithrombin deficiency | SERPINC1 |
| Deep vein thrombosis | SERPINC1 |
| Angioneurotic oedema | SERPING1 |
| Surfactant protein B deficiency | SFTPB |
| Muscular dystrophy, limb girdle | SGCD |
| Myoclonus dystonia | SGCE |
| Muscular dystrophy, limb girdle | SGCG |
| Sanfilippo syndrome A | SGSH |
| Lymphoproliferative syndrome, X-linked | SH2D1A |
| Holoprosencephaly | SHH |
| Leri-Weill dyschondrosteosis | SHOX |
| JK-null variant | SLC14A1 |
| Cataract, juvenile with microcornea and renal glucosuria | SLC16A12 |
| Monocarboxylate transporter 8 deficiency | SLC16A2 |
| Salla disease | SLC17A5 |
| Sialic acid storage disease, infantile | SLC17A5 |
| Megaloblastic anaemia, thiamine responsive | SLC19A2 |
| Organic cation transporter deficiency | SLC22A4 |
| Intrahepatic cholestasis, neonatal | SLC25A13 |
| HHH syndrome | SLC25A15 |
| Diarrhoea, congenital chloride | SLC26A3 |
| Glucose transporter 1 deficiency syndrome | SLC2A1 |
| Fanconi-Bickel syndrome | SLC2A2 |
| Hereditary hypophosphataemic rickets with hypercalciuria | SLC34A3 |
| Acrodermatitis enteropathica | SLC39A4 |
| Cystinuria | SLC3A1 |
| Spherocytosis | SLC4A1 |
| Corneal endothelial dystrophy 2 | SLC4A11 |
| Proximal renal tubular acidosis | SLC4A4 |
| Glucose/galactose malabsorption | SLC5A1 |
| Renal glucosuria | SLC5A2 |
| Iodide transport defect | SLC5A5 |
| Hyperekplexia | SLC6A5 |
| Creatine deficiency | SLC6A8 |
| Lysinuric protein intolerance | SLC7A7 |
| Cystinuria, type I/III | SLC7A9 |
| Mal de Meleda | SLURP1 |
| Juvenile polyposis syndrome | SMAD4 |
| Pulmonary arterial hypertension | SMAD9 |
| Schimke immuno-osseous dysplasia | SMARCAL1 |
| Schimke immuno-osseous dysplasia | SMARCAL1 |
| Spinal muscular atrophy | SMN1 |
| Niemann-Pick disease | SMPD1 |
| Amyotrophic lateral sclerosis | SOD1 |
| Sclerosteosis | SOST |
| PCWH | SOX10 |
| Shah-Waardenburg syndrome and neuropathy | SOX10 |
| Hypotrichosis-Lymphoedema-Telangiectasia | SOX18 |
| Anophthalmia, hearing loss and brain abnormalities | SOX2 |
| Anophthalmia-oesophageal-genital syndrome | SOX2 |
| Campomelic dysplasia | SOX9 |
| Spastic paraplegia | SPAST |
| Spastic paraplegia, autosomal dominant | SPAST |
| Retiniitis pigmentosa, juvenile | SPATA7 |
| Leber congenital amaurosis IV | SPATA7 |
| Spastic paraplegia, autosomal recessive | SPG11 |
| Spastic paraplegia with thin corpus callosum | SPG11 |
| Netherton syndrome | SPINK5 |
| Neurofibromatosis 1-like syndrome | SPRED1 |
| Legius syndrome | SPRED1 |
| Cafe-au-lait macules | SPRED1 |
| Pyropoikilocytosis | SPTA1 |
| Spherocytosis | SPTB |
| Steroid-5 alpha-reductase deficiency | SRD5A2 |
| XY sex reversal | SRY |
| Gonadal dysgenesis | SRY |
| Amish infantile epilepsy syndrome | ST3GAL5 |
| Congenital lipoid adrenal hyperplasia | STAR |
| Growth hormone insensitivity | STAT5B |
| Gonadotrophin-independent precocious puberty | STK11 |
| Peutz-Jeghers syndrome | STK11 |
| Microphthalmia | STRA6 |
| Haemophagocytic lymphohistiocytosis, familial | STX11 |
| Glutaric aciduria 3 | SUGCT |
| Sulphite oxidase deficiency | SUOX |
| Leigh syndrome | SURF1 |
| Epilepsy | SYN1 |
| Schizophrenia | syngr1c |
| Corneal dystrophy, gelatinous drop-like | TACSTD2 |
| Tyrosinaemia 2 | TAT |
| Barth syndrome | TAZ |
| Cardiomyopathy, X-linked infantile | TAZ |
| Amyotrophic lateral sclerosis | TBK1 |
| ACTH deficiency, isolated | TBX19 |
| Congenital heart disease | TBX20 |
| Cleft palate and ankyloglossia | TBX22 |
| Ulnar-mammary syndrome | TBX3 |
| Holt-Oram syndrome | TBX5 |
| Osteopetrosis, autosomal recessive | TCIRG1 |
| Transcobalamin II deficiency | TCN2 |
| Treacher-Collins syndrome | TCOF1 |
| Haemochromatosis | TFR2 |
| Goitre with hypothyroidism | TG |
| Goitre, simple | TG |
| Holoprosencephaly | TGIF1 |
| Ichthyosis, congenital, autosomal recessive | TGIF1 |
| Ichthyosis, lamellar | TGM1 |
| Dystonia | THAP1 |
| Thyroid hormone resistance | THRB |
| Epidermodysplasia verruciformis | TMC6 |
| Epidermodysplasia verruciformis | TMC8 |
| Enteropeptidase deficiency | TMPRSS15 |
| Microcytic anaemia & iron deficiency | TMPRSS6 |
| Microcytic anaemia & iron deficiency | TMPRSS6 |
| Li-Fraumeni syndrome | TP53 |
| Multiple cancers | TP53 |
| Osteosarcoma | TP53 |
| Adrenocortical carcinoma | TP53 |
| Split-hand/split-foot malformation | TP63 |
| Nemaline myopathy | TPM3 |
| Goitrous hypothyroidism | TPO |
| Neuronal ceroid lipofuscinosis, late infantile | TPP1 |
| Spondyloepiphyseal dysplasia tarda | TRAPPC2 |
| Deafness, non-syndromic | TRIOBP |
| Hypomagnesaemia with secondary hypocalcaemia | TRPM6 |
| Tricho-rhino-phalangeal syndrome I | TRPS1 |
| Tuberous sclerosis | TSC1 |
| Tuberous sclerosis | TSC2 |
| Hypothyroidism | TSHB |
| Hyperthyroidism | TSHR |
| Tibial muscular dystrophy | TTN |
| Cardiomyopathy, dilated | ttntvn2b |
| Saethre-Chotzen syndrome | TWIST1 |
| Baller-Gerold syndrome | TWIST1 |
| Albinism, oculocutaneous 1 | TYR |
| Albinism, oculocutaneous 1A | TYR |
| Albinism, oculocutaneous 3 | TYRP1 |
| Hypotrichosis, Marie Unna type | u2hr |
| Angelman syndrome | UBE3A |
| Crigler-Najjar syndrome 1 | UGT1A1 |
| Crigler-Najjar syndrome 2 | UGT1A1 |
| Haemophagocytic lymphohistiocytosis, familial | UNC13D |
| Mental retardation | UPF3B |
| Porphyria, hepatoerythropoietic | UROD |
| Porphyria, cutanea tarda | UROD |
| Porphyria, erythropoietic | UROS |
| Usher syndrome 1c | USH1C |
| Usher syndrome 1g | USH1G |
| Usher syndrome 2a | USH2A |
| Retinitis pigmentosa, recessive, no hearing loss | USH2A |
| Usher syndrome 2 | USH2A |
| Rickets, vitamin D resistant | VDR |
| Von Hippel-Lindau syndrome | VHL |
| Cerebellar hypoplasia and quadrupedal locomotion | VLDLR |
| Dysequilibrium syndrome | VLDLR |
| Chorea-acanthocytosis | VPS13A |
| Cohen syndrome | VPS13B |
| Von Willebrand disease 3 | VWF |
| Von Willebrand disease | VWF |
| Von Willebrand disease 2n | VWF |
| Wiskott-Aldrich syndrome | WAS |
| Wolfram syndrome | WFS1 |
| Neuropathy, hereditary sensory, type II | wnk1tv3 |
| Odonto-onycho-dermal dysplasia | WNT10A |
| Tetra-amelia | WNT3 |
| Werner syndrome | WRN |
| Wilms tumour | WT1 |
| Renal dysfunction & renal blastema | WT1 |
| Xanthinuria, type 1 | XDH |
| Xeroderma pigmentosum (A) | XPA |
| Xeroderma pigmentosum (C) | XPC |
| Posterior polymorphous corneal dystrophy | ZEB1 |
| Mowat-Wilson syndrome | ZEB2 |
| Cardiac malformation | ZIC3 |
| Situs abnormality | ZIC3 |
| Mental retardation, X-linked | ZNF674 |

TABLE 2
Short List of Medical Conditions Associated with PTC

| Medical Condition Associated with PTC | Gene symbol |
|---|---|
| Muscular Dystrophy (Duchenne or Becker) | DMD |
| Chronic granulomatous disease | CYBB or NCF1 or NCF2 |
| Late infantile neuronal ceroid lipofuscinosis | TPP1 |
| Neuronal ceroid lipofuscinosis (juvenile, infantile or late | PPT1 |
| infantile) | |
| Neuronal ceroid lipofuscinosis (juvenile or late or late | CLN3, CLN5, CLN6, or |
| infantile) | MFSD8 |
| Frontotemporal dementia | GRN or CHMP2B |
| Epidermolysis bullosa (dystrophic/dystrophica, | COL7A1 or COL17A1 |
| junctional, atrophic benign) | |
| Rett syndrome | MECP2 |
| Congenital disorder of deglycosylation (IIh or 1a) | COG8 or PMM2 or NGLY1 |
| Cystic fibrosis | CFTR |
| Schimke immuno-osseous dysplasia | SMARCAL1 |
| Adenomatous polyposis coli | APC |
| Li-Fraumeni syndrome | TP53 |
| Sporadic cancer | various tumour suppressor |
| | genes including TP53 |

The codon changes resulting in all of the above medical conditions are well known in the art and new codon changes that result in PTC are still being discovered. Nevertheless, there is an expectation that the compounds described herein will have some degree of readthrough activity for all such PTCs.

Compounds as described herein may be in the free form or in the form of a salt thereof. In some embodiment, compounds as described herein may be in the form of a pharmaceutically acceptable salt, which are known in the art (Berge S. M. et al., J. Pharm. Sci. (1977) 66(1):1-19). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

A PTC read-through compound may provide a therapeutic benefit if the compound permits read-through of a PTC in a protein coding sequence to produce the full length protein. Wherein the full length protein may have sequence variations and may not be the same as the native protein. Generally, the full length protein produced by the read-through is functional and can stand in for the wild-type protein. In some cases, as little as 5% of the normal total amount of the full length protein, wherein the total amount of protein, is what a subject not having the medical condition associated with the PTC would normally produce. However, depending on the medical condition associated with the PTC, as little as 1% of the normal total amount of the full length protein may be sufficient to have a therapeutic benefit. Provided that the PTC read-through compound provides read-through of a PTC in a protein coding sequence to produce at least about 1% of the normal total amount of the full length protein a therapeutic benefit may be achieved. Provided that the PTC read-through compound provides read-through of a PTC in a protein coding sequence to produce at least about 2% of the normal total amount of the full length protein a therapeutic benefit may be achieved. Provided that the PTC read-through compound provides read-through of a PTC in a protein coding sequence to produce at least about 3% of the normal total amount of the full length protein a therapeutic benefit may be achieved. Provided that the PTC read-through compound provides read-through of a PTC in a protein coding sequence to produce at least about 4% of the normal total amount of the full length protein a therapeutic benefit may be achieved. Provided that the PTC read-through compound provides read-through of a PTC in a protein coding sequence to produce at least about 5% of the normal total amount of the full length protein a therapeutic benefit may be achieved. Provided that the PTC read-through compound provides read-through of a PTC in a protein coding sequence to produce at least about 6% of the normal total amount of the full length protein a therapeutic benefit may be achieved. Provided that the PTC read-through compound provides read-through of a PTC in a protein coding sequence to produce at least about 7% of the normal total amount of the full length protein a therapeutic benefit may be achieved. Provided that the PTC read-through compound provides read-through of a PTC in a protein coding sequence to produce at least about 8% of the normal total amount of the full length protein a therapeutic benefit may be achieved. Provided that the PTC read-through compound provides read-through of a PTC in a protein coding sequence to produce at least about 9% of the normal total amount of the full length protein a therapeutic benefit may be achieved. Provided that the PTC read-through compound provides read-through of a PTC in a protein coding sequence to produce at least about 10% of the normal total amount of the full length protein a therapeutic benefit may be achieved.

Alternatively, a PTC read-through compound may provide a therapeutic benefit if the compound permits sufficient read-through of a PTC in a protein coding sequence to provide some therapeutic benefit to the subject or achieve some therapeutic result. The therapeutic benefit may be determined functionally by measuring some therapeutic result. A therapeutic result may result from a therapeutically effective amount or a prophylactically effective amount of the compound, and may include, for example, reduced tumor size, increased life span, a delay of symptom onset or disease onset, increase metabolic efficiency or increased life expectancy. A therapeutically effective amount of a compound or a prophylactically effective amount of a compound may vary according to the disease state, age, sex, other health factors unrelated to or related to the disease and weight of the subject, and the ability of the compound to elicit a desired response in the subject.

Furthermore, the read-through efficiently may be greater at TGA than TAG, and in some circumstances there may be no read-through at TAA. Accordingly, treatments may be tailored to particular stop codons.

In some embodiments, compounds and all different forms thereof (e.g. free forms, salts, solvates, polymorphs, protonated forms) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

For example, Gentamicin B1 may be represented as follows:

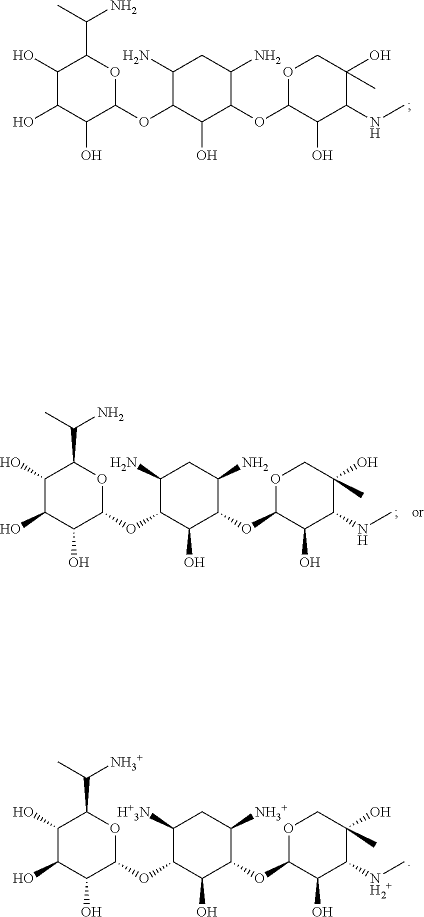

In some embodiments, compounds may include analogs, isomers, stereoisomers, or related derivatives. In some embodiments the compounds may be used in conjunction with another compound to form a pharmaceutical composition.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result (for example, smaller tumors, increased life span, increased life expectancy or prevention of the progression of the medical condition associated with premature termination codons). Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In some embodiments, compounds and all different forms thereof as described herein may be used, for example, and without limitation, in combination with other treatment methods for at least one indication selected from the group set out in TABLE 1 or TABLE 2.

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Toxicity may be evaluated by examining a particular compound's or composition's specificity across cell lines or in an animal model.

Compounds as described herein may be administered to a subject. As used herein, a “subject” may be a human, non-human primate, rat, mouse, cow, horse, pig, sheep, goat, dog, cat, etc. The subject may be suspected of having or at risk of having a medical condition associated with premature termination codons (PTCs).

As used herein, a “medical condition associated with premature termination codons” may be defined as any medical condition caused in whole or in part by a nonsense codon, which may result in decreased mRNA stability as well as protein truncation resulting in a non-functional protein, which in turn may directly or indirectly result in the medical condition. For example, the medical condition associated with premature termination codons may be selected from TABLE 1 or TABLE 2.

There are about 5000 or so such genetic diseases which may be grouped into broad categories, as follows: an autoimmune disease; a blood disease; a collagen disease; diabetes; a neurodegenerative disease; a cardiovascular disease; a pulmonary disease; or an inflammatory disease; a neoplastic disease or central nervous system disease. One third of the cases of genetic inherited diseases involve a premature termination codon (PTC) (Frischmeyer P A and Dietz H C 1999). In most cases, the primary mechanism whereby a nonsense mutation has an effect is through the degradation of that mRNA by a surveillance mechanism called nonsense-mediated mRNA decay (NMD) (see: Chang Y F et al. 2007; Isken O and Maquat L E 2007; Rebbapragada I and Lykke-Andersen J 2009; Rehwinkel J et al. 2006; and Muhlemann 0 et al. 2008).

Diagnostic methods for various medical conditions associated with premature termination codons are known in the art. Depending on the condition genetic diagnostics may be readily available or may be determined with directed sequencing. For example, the medical condition may be selected from the group consisting of central nervous system diseases, ataxia-telangiectasia, muscular dystrophy, Duchenne muscular dystrophy, Dravet syndrome, myotonic dystrophy, multiple sclerosis, infantile neuronal ceroid lipofuscinosis, Alzheimer's disease, Tay-Sachs disease, neural tissue degeneration, Parkinson's disease, autoimmune diseases, chronic rheumatoid arthritis, lupus erythematosus, graft-versus-host disease, primary immunodeficiencies, severe combined immunodeficiency, DNA Ligase IV deficiency, DNA repair disorders, Nijmegen breakage disorders, xeroderma pigmentosum (XP), inflammatory diseases, rheumatoid arthritis, blood diseases, hemophilia, von Willebrand disease, thalassemia (for example, β-thalassemia), familial erythrocytosis, nephrolithiasis, collagen diseases, osteogenesis imperfecta, cirrhosis, neurofibroma, bullous disease, lysosomal storage disease, Hurler's disease, familial cholesterolemia, cerebellar ataxia, tuberous sclerosis, immune deficiency, kidney disease, lung disease, cystic fibrosis, familial hypercholesterolemia, pigmentary retinopathy, retinitis pigmentosa, amyloidosis, atherosclerosis, giantism, dwarfism, hypothyroidism, hyperthyroidism, aging, obesity, diabetes mellitus, familial polycythemia, Niemann-Pick disease, epidermolysis bullosa, Marfan syndrome, neuromuscular diseases, Becker muscular dystrophy (BMD), spinal muscular atrophy, cancer, and any genetic disorder caused by nonsense mutation(s). Furthermore, where the medical condition associated with a premature termination codon is a cancer, the cancer may be selected from one or more of cancer is of the head and neck, eye, skin, mouth, throat, esophagus, chest, bone, blood, lung, colon, sigmoid, rectum, stomach, prostate, breast, ovaries, kidney, liver, pancreas, brain, intestine, heart or adrenals. Alternatively, the cancer may be selected from sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor or multiple myeloma. Tests for determining whether a PTC is involved in the condition are known to those of ordinary skill in the art.

Compounds tested and to be tested are set out below in TABLES A and B respectively.

TABLE A

| Compound | | PTC Read- |
|---|---|---|
| Identifier | Structure | through |
| Gentamicin B1 or B1 | 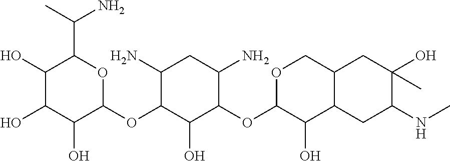 [SMILES: CNC1CC2C(COC(OC3C(N)CC(N)C(OC4OC(C(C)N)C(O)C(O)C4O)C3O)C2O)CC1(C)O] | Y |
| G-418 or G418 | 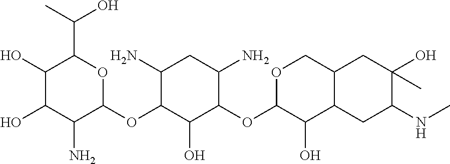 [SMILES: CNC1CC2C(COC(OC3C(N)CC(N)C(OC4OC(C(C)O)C(O)C(O)C4N)C3O)C2O)CC1(C)O] | Y |
| Gentamicin X2 or X2 | 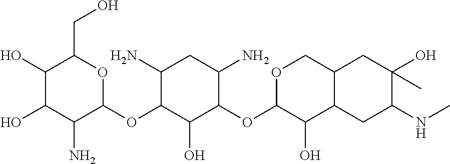 [SMILES: CNC1CC2C(COC(OC3C(N)CC(N)C(OC4OC(CO)C(O)C(O)C4N)C3O)C2O)CC1(C)O] | Y |

TABLE B

| Compound | | PTC Read- |
|---|---|---|
| Identifier | Structure | through |
| JI-20B CAS 51846-98-1 | 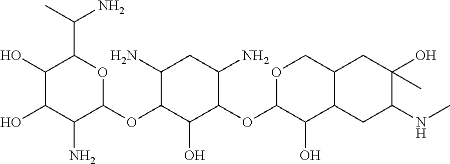 [SMILES: CNC1CC2C(COC(OC3C(N)CC(N)C(OC4OC(C(C)N)C(O)C(O)C4N)C3O)C2O)CC1(C)O] | Not yet tested |
| Analogue A | 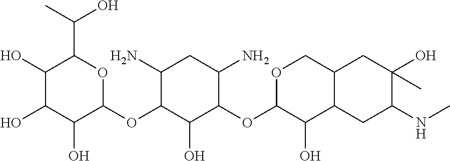 [SMILES: CNC1CC2C(COC(OC3C(N)CC(N)C(OC4OC(C(C)O)C(O)C(O)C4O)C3O)C2O)CC1(C)O] | Not yet tested |
| CAS# 52945-42-3 | 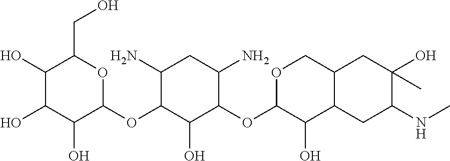 [SMILES: CNC1CC2C(COC(OC3C(N)CC(N)C(OC4OC(CO)C(O)C(O)C4O)C3O)C2O)CC1(C)O] | Not yet tested |

The Gentamicin complex or Gentamicin C complex as used herein includes gentamicin C1, gentamicin C1a, and gentamicin C2 (˜80% of complex) and are reported to have the most significant antibacterial activity. The remaining ˜20% of the complex is made up of Gentamicins A, B, X, et al. The exact compositions may vary between different production runs and based on the producer.

Various alternative embodiments and examples of the invention are described herein. These embodiments and examples are illustrative and should not be construed as limiting the scope of the invention.

### Materials and Methods

### p53 PTC Read-Through in a Human Cell Line.

Compounds were tested for PTC read-through in human cells, wherein mammary carcinoma HDQ-P1 cells homozygous for TGA (R213X) in exon 6 of the TP53 gene (Wang et al., 2000) were selected on the basis of convincing evidence of read-through by the aminoglycoside G418 (Floquet, C. et al. 2011). Western analysis using a quantitative automated capillary electrophoresis system showed that HDQ-P1 cells express very low levels of truncated p53 and no full-length p53 and that 50 μM G418 induces the formation of full-length p53 while also increasing truncated p53 levels as reported (Floquet, C. et al. 2011).

Nuclear localization sequences and a tetramerization domain located in the p53 C-terminus contribute to retaining p53 in the nucleus (Shaulsky, G et al. 1990; Liang, S. H and Clark, M. F. 2001) and p53 truncated at R213 lacks these sequences. To enable analysis of p53 R213X read-through at high throughput an automated 96-well fluorescence microscopy assay was established to detect and quantitate nuclear p53 signal. G418 induced a concentration-dependent increase in nuclear p53 consistent with read-through induction. During 72 h exposure, 50 μM G418 induced nuclear 53 expression in 9% of cells while 250 μM G418 induced nuclear p53 expression in nearly all cells.

### Automated p53 Immunofluorescence 96-Well Plate Assay

HDQ-P1 cells cultured in DMEM containing 10% FBS and 1× Gibco™ antibioticantimicotic were seeded at 4000 per well of PerkinElmer View™ 96-well plates. The next day, the medium was replaced with fresh culture medium containing the compounds to be tested. After 72 h, the culture medium was removed by aspiration, the cells were fixed with 3% paraformaldehyde, 0.3% Triton X-100 and 1.5 μg/ml Hoechst 33323 in phosphate-buffered saline pH 7.2 (PBS) for 20 min at room temp. The cells were rinsed once with PBS and incubated for 2 h at room temp with a blocking solution of 3% BSA in PBS. The blocking solution was removed by aspiration and cells were incubated with 0.1 μg/ml DO-1 p53 mouse monoclonal p53 antibody (Santa Cruz™) in blocking solution for 90 min at room temp. The wells were washed once with PBS for 5 min and the cells were incubated with Alexa 488-conjugated goat anti-mouse antibody (Invitrogen Life Technologies A11029™) in blocking buffer for 90 min at room temp. The wells were washed once with PBS for 5 min, 75 μl PBS was added, the plates were covered with a black adherent membrane and stored at 4° C. overnight. Nuclear p53 immunofluorescence intensity was measured using a Cellomics ArrayScan VTI™ automated fluorescence imager.

Briefly, images were acquired with a 20× objective in the Hoechst™ and GFP (XF53) channels. Images of 15 fields were acquired for each well, corresponding to ˜2000 cells.

The Compartment Analysis bioapplication was used to identify the nuclei and define their border. The nuclear Alexa 488™ fluorescence intensity was then measured and expressed as average nuclear fluorescence intensity or % positive nuclei, using as a threshold the fluorescence intensity of nuclei from untreated cells (50-75, depending on experiment).

### Automated Electrophoresis Western Analysis Assay

HDQ-P1 cells were seeded at 100,000 cells per well of TC-treated 6-well plates. The next day, the medium was replaced with fresh medium containing compounds to be tested and were incubated for 48 to 96 h. The medium was removed by aspiration, cell monolayers were rinsed with 1 ml ice-cold PBS. Cells were lysed in 80 μl lysis buffer (20 mM Tris-HCl pH 7.5, 150 mM NaCl, 1 mM EDTA, 1 mM EGTA, 1% (v/v) Triton X100™, 2.5 mM sodium pyrophosphate, 1 mM beta-glycerophosphate supplemented with fresh 1 mM Na₃VO₄, 1 mM dithiothreitol and 1× complete protease inhibitor cocktail (Roche Molecular Biochemicals™)). Lysates were pre-cleared by centrifugation at 18,000 g for 15 min at 4° C. Supernatants were collected, protein was quantitated using the Bradford assay and lysates were adjusted to 1 mg/ml protein. Capillary electrophoresis and western analysis conditions were carried out with manufacturer's reagents according to the user manual (ProteinSimple WES™). Briefly, 5.6 μl of cell lysate was mixed with 1.4 μl fluorescent master mix and heated at 95° C. for 5 min. The samples, blocking reagent, wash buffer, DO-1 p53 antibody (0.5 μg/ml) and vinculin antibody (1:2000, R&D clone 728526), secondary antibody and chemiluminescent substrate were dispensed into the microplate provided by the manufacturer. The electrophoretic separation and immunodetection was performed automatically using default settings. The data was analyzed with inbuilt Compass™ software (Proteinsimple™). The truncated and full-length p53 peak intensities were normalized to that of the vinculin peak, used as a loading control. Results are shown as pseudo blots and as electropherograms.

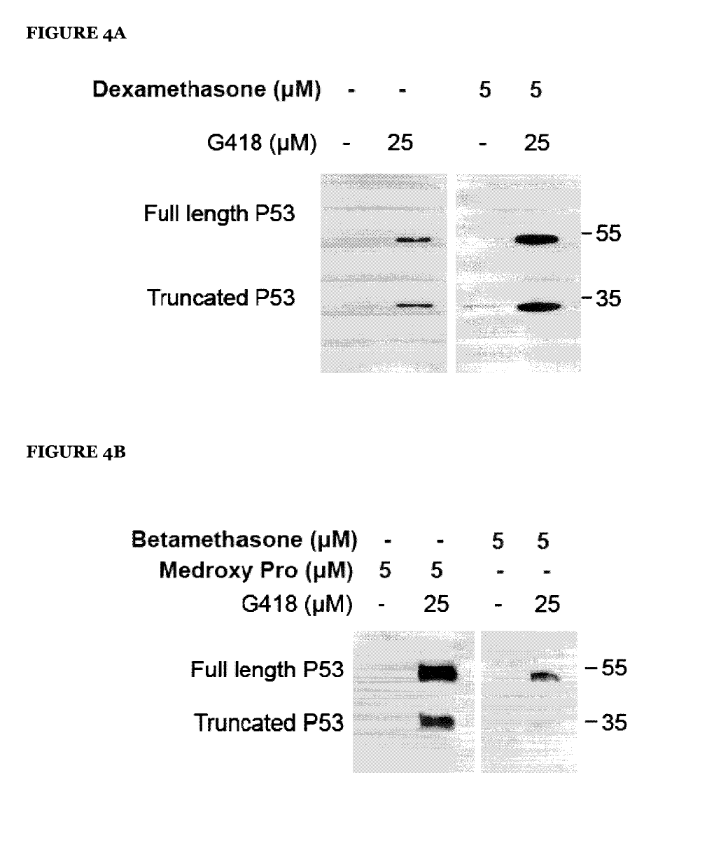

In some instances, a traditional western blotting procedure was used (e.g. FIG. 4), using the same antibodies.

### Compounds Tested

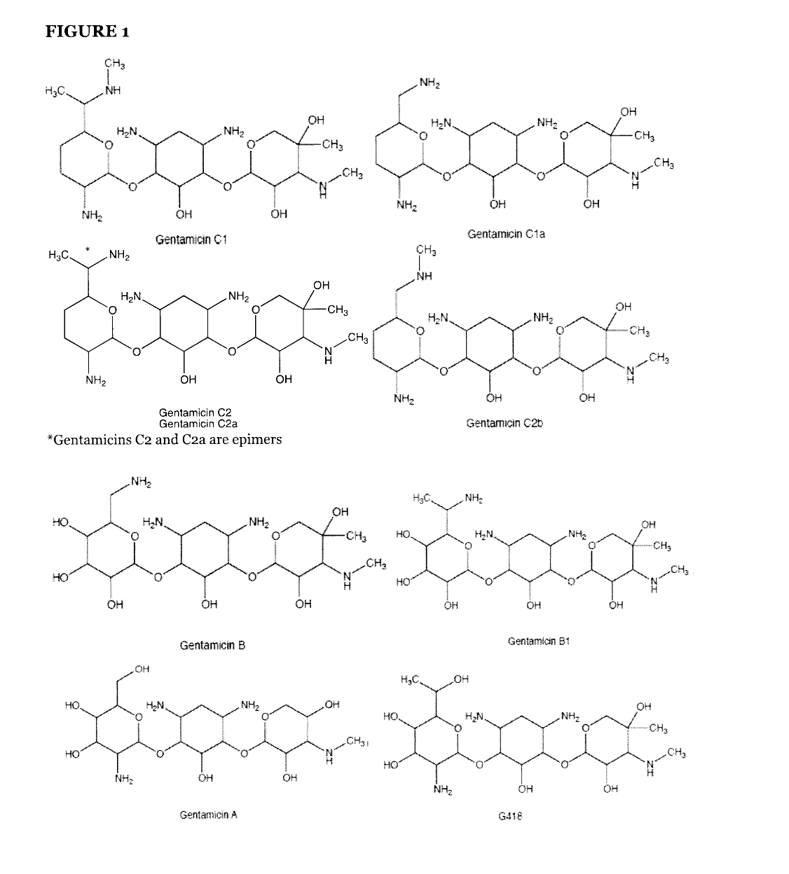

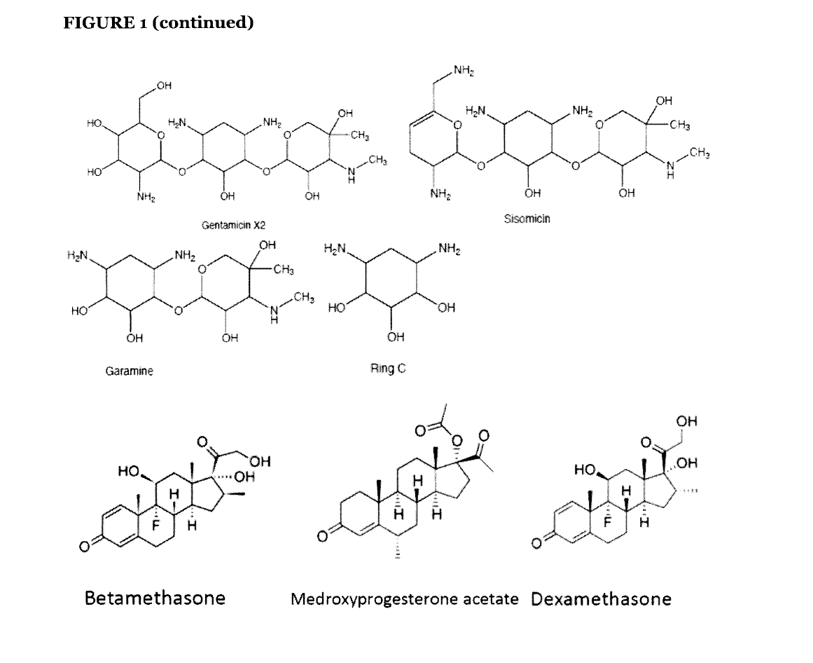

Gentamicin, gentamicin A, B, B1, C1, C1a, C2, C2a, C2b, X2, sisomicin, as well as gentamicin fragments garamine and ring C (see FIG. 1) were obtained from MicroCombiChem. G418 was from Sigma™. Betamethasone, dexamethasone and medroxyprogesterone acetate were from Sigma™. Gentamicin B1 was purchased from MicroCombiChem™ (catalogue # MCC3436). Gentamicin X2 was from TokuoE (catalogue # G036). G418 from Life Technologies (catalogue #11811-023). Gentamicin from Sigma (catalogue # G1264).

### Immunofluorescence P53 Testing

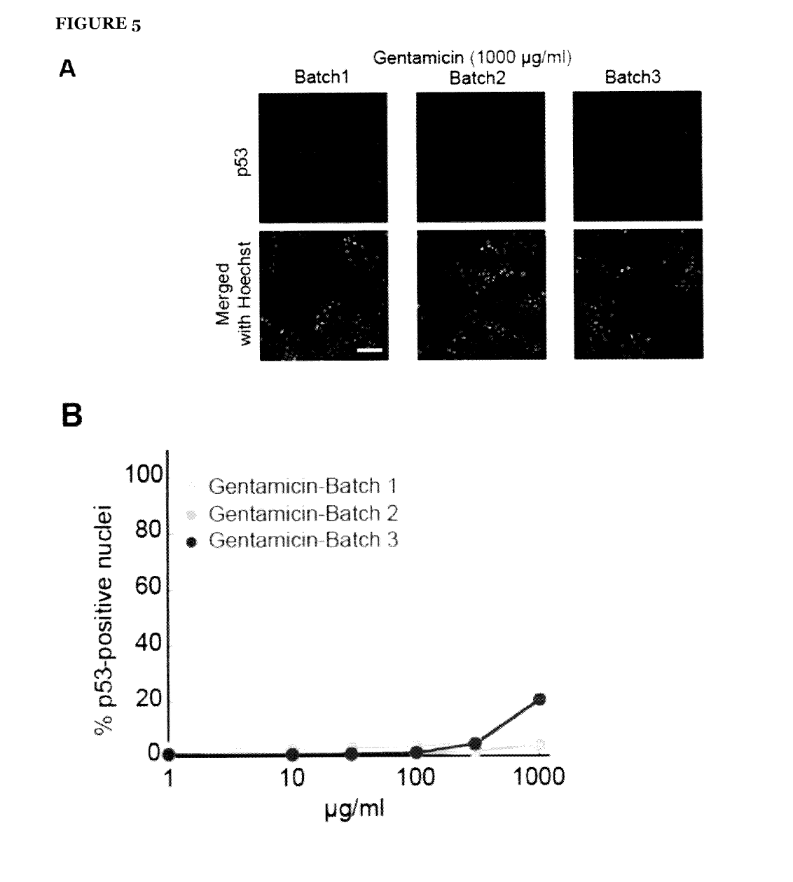

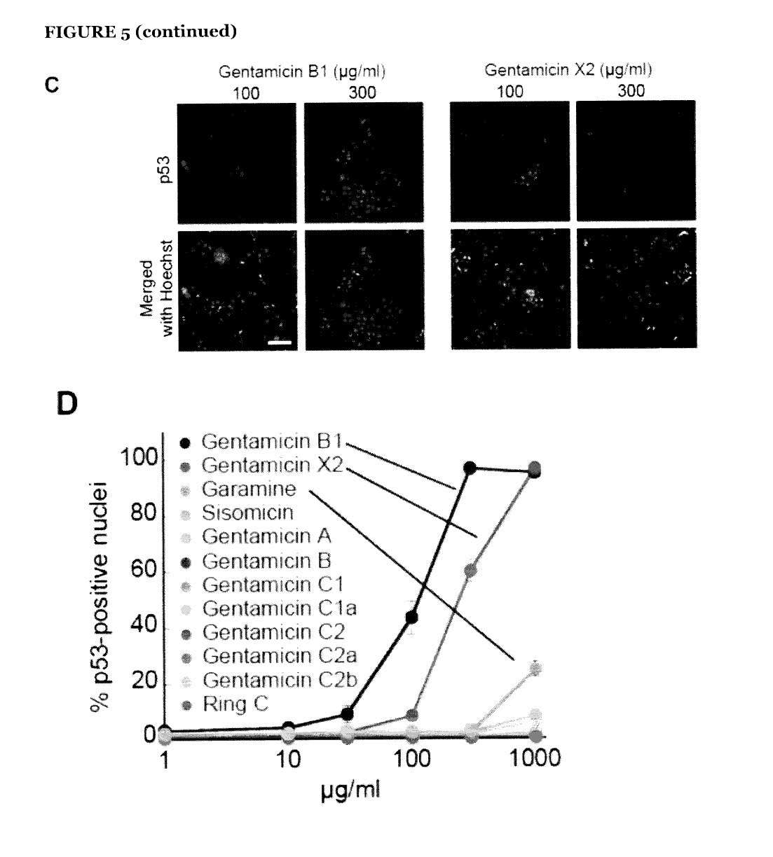

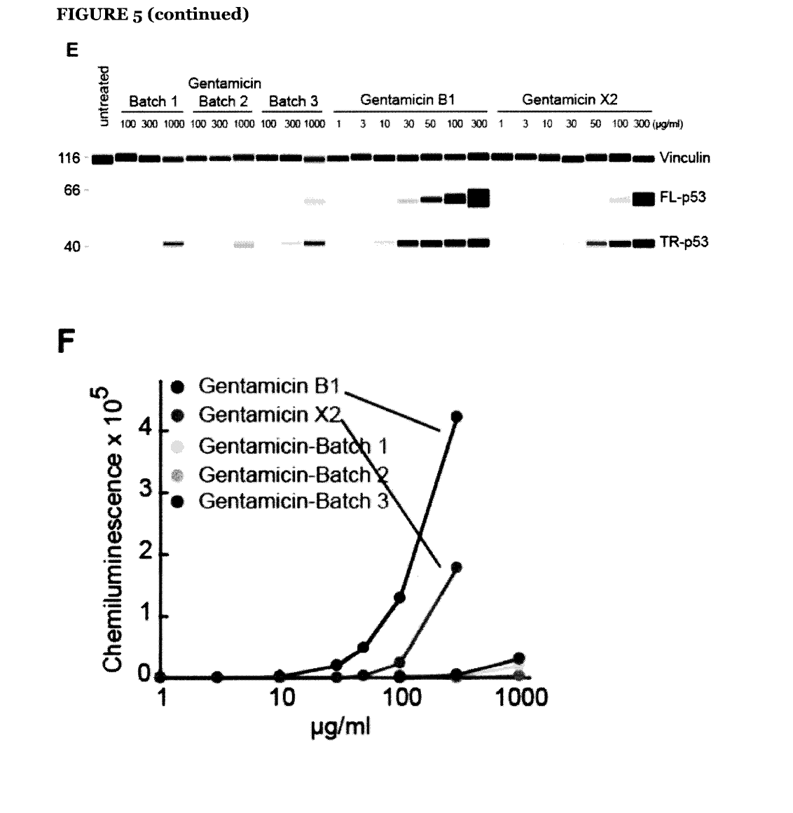

Methods for FIG. 5: Panels A to D: Human HDQ-P1 breast carcinoma cells with a homozygous R213× nonsense mutation in the TP53 gene were exposed to three different batches of pharmaceutical gentamicin sulfate or to major and minor gentamicin components purified from pharmaceutical gentamicin, for 72 h. The cells were then fixed, DNA was stained with Hoechst™ 33323 and nuclear p53 was detected by immunofluorescence labeling using Santa Cruz DO-1 p53 antibody. The p53-positive nuclei were determined using a Cellomics™ VTI 96-well imager as described in Baradaran-Heravi et al. (2016). The percent p53-positive nuclei is a measure of the extent of PTC readthrough. Panels E and F: HDQ-P1 cells were exposed to the gentamicin batches, gentamicin B1 or gentamicin X2 for 72 h and subjected to p53 Western analysis using Santa Cruz™ DO-1 p53 antibody as described in Baradaran-Heravi et al. (2016) to measure formation of truncated p53 and full-length p53, where full-length p53 is the PTC readthrough product. The y axis in Panel F shows the full-length p53 signal intensity, expressed as chemiluminescence units.

Premature Stop Codon Testing with Genatamicin B1

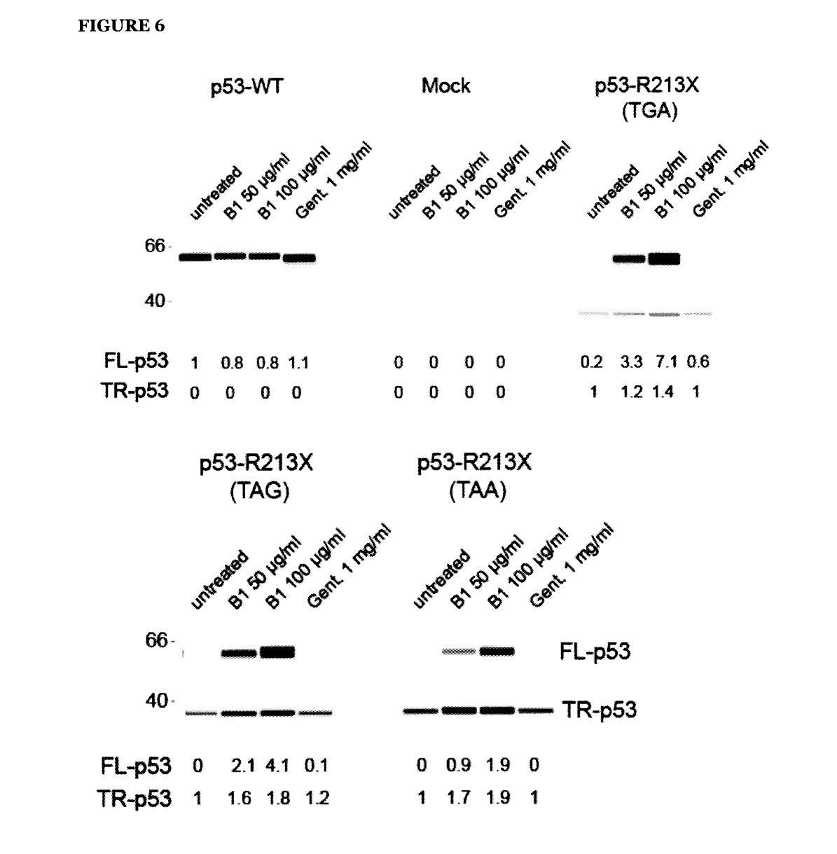

Methods for FIG. 6: NCI-H1299 human non-small cell lung carcinoma cells were transiently transfected with p53 expression constructs bearing a TGA, TAG or TAA nonsense mutation at amino acid position 213. Cells exposed to transfection reagent only (mock) or transiently transfected with a WT p53 expression were included as controls. The cells were exposed to the indicated concentrations of gentamicin B1 or USP gentamicin sulfate (Sigma™) for 48 h and the formation of truncated p53 and full-length p53 (readthrough product) was determined as described in Baradaran-Heravi et al. (2016). The amounts of full-length p53 and truncated p53 are expressed relative to the amount of full-length (for WT) or truncated p53 in untreated cells.

Premature Stop Codon Testing with Genatamicin B1

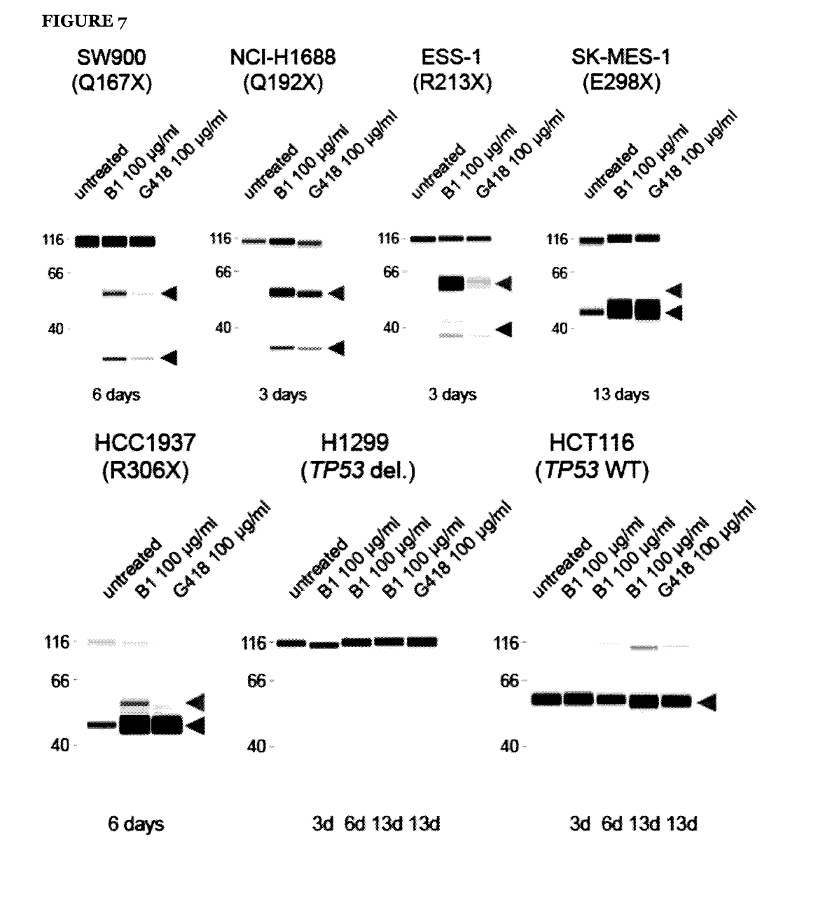

Methods for FIG. 7: Different human cancer cell lines with homozygous TP53 nonsense mutations (i.e. SW900; NCI-H1688; ESS-1; SK-MES-1; HCC1937; H1299; and HCT116) were exposed to the indicated concentrations of gentamicin B1 or G418 for 3 days, 6 days or 13 days, as indicated and the formation of truncated p53 (lower arrowhead) and full length p53 (upper arrowhead, readthrough product) was determined as described in Baradaran-Heravi et al. (2016). The nonsense mutations are indicated under the cell line names. Vinculin, which migrates around 116 kDa, was used as a protein loading control.

### Induction of PTC Readthrough In Vivo

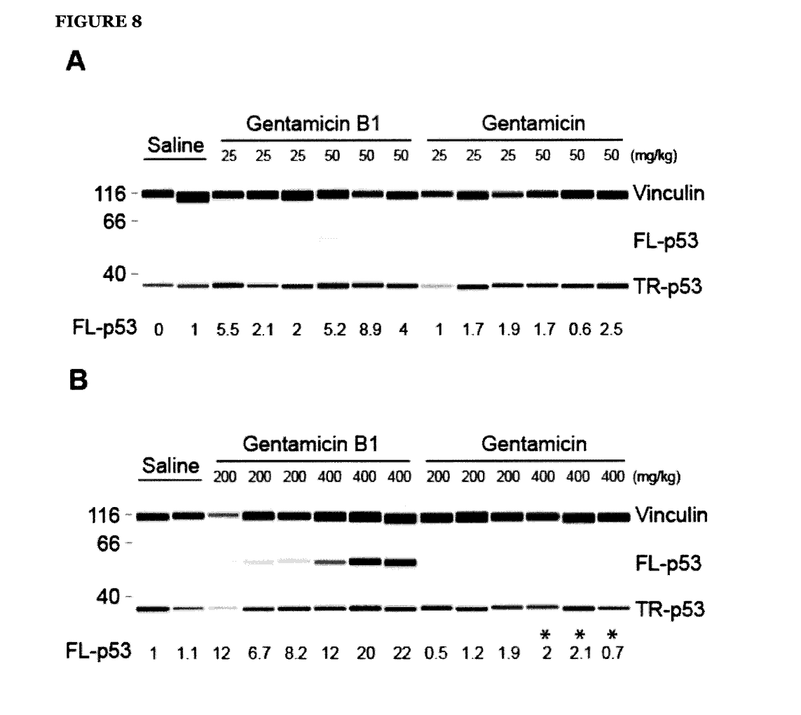

Methods for FIG. 8: Two million NCI-H1299 human non-small cell lung carcinoma cells stably expressing a TP53 expression construct bearing the R213X (TGA) nonsense mutation were implanted subcutaneously on the lower back of immunocompromised NRG (NOD-Rag1^{null} IL2rg^{null}) mice. Panel A: When the tumour xenografts reached a size of approximately 0.2-0.5 cubic centimeters, the mice were injected intraperitoneally with saline, gentamicin B1 or USP gentamicin sulfate at the indicated doses for 5 consecutive days. 72 hours after the last injection, the mice were sacrificed and the amounts of truncated p53 (TR-p53) and full-length p53 (FL-p53) were determined by western analysis as described in Baradaran-Heravi et al. (2016). Panel B: When the tumour xenografts reached a size of approximately 0.2-0.5 cubic centimeters, the mice were injected intraperitoneally once with saline, gentamicin B1 or USP gentamicin sulfate. 48 hours after the last injection, the mice were sacrificed and the amounts of truncated p53 and full-length p53 were determined by western analysis. The amounts of full-length p53 relative to saline-treated mice are indicated under each lane. Vinculin was used as a protein loading control.

Induction of PTC Readthrough by Gentamicin B1 in Cells Derived from Patients with Rare Genetic Diseases.

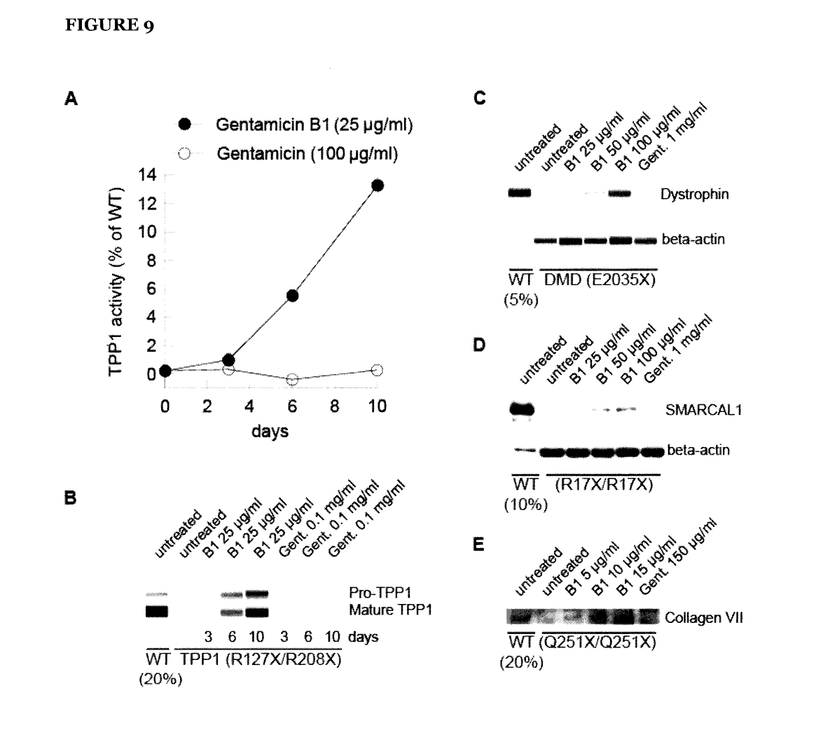

Methods for FIG. 9: Panels A and B: GM16485 primary fibroblasts derived from a Neuronal Ceroid Lipofuscinosis patient with compound heterozygous nonsense mutations in the TPP1 (tripeptidylpeptidase I) gene (R127X/R208X) were exposed to 25 μg/ml gentamicin B1 or 100 μg/ml gentamicin for up to 10 days. Cell lysates were prepared and TPP1 enzyme activity was determined as in Lojewski et al. (2014) with modifications: Lysates were diluted 1:5 in 50 mM sodium acetate pH 4.0 and pre-incubated at 37° C. for 1 h. After pre-incubation, 2014 of total protein from GM16485 lysates or 5 μg of total protein from lysates of fibroblasts from unaffected individuals (WT) was incubated in 150 μl of 50 mM sodium acetate pH 4.0 containing a final concentration of 62.5 μM Ala-Ala-Phe-7-amido-4-methylcoumarin for 2 h at 37° C. Fluorescence was measured using a TECAN Infinite M200™ spectrophotometer with an excitation wavelength of 360 nm and an emission wavelength of 460 nm. Assays were carried out under conditions where product formation was linear with respect to protein concentration and time. TPP1 activity was expressed relative to the average activity of untreated primary fibroblasts from two unaffected individuals (WT) (Panel A). For panel B, the same cell extracts were analysed for formation of TPP1 by automated capillary electrophoresis western analysis using the Abcam™ ab54685 α-TPP1 antibody as in Baradaran-Heravi et al (2016). Extracts from WT fibroblasts were also analysed, using 20% of the amount of protein used for GM16485.

Panel C: HSK001 myoblasts derived from a Duchenne Muscular Dystrophy patient with nonsense mutation (DMD: E2035X) were differentiated into myotubes and exposed to the indicated concentrations of gentamicin B1 or gentamicin for 3 days and dystrophin expression level was determined by automated capillary electrophoresis western analysis using Abeam™ ab1527 α-dystrophin antibody. Extracts from WT myotubes were also analyzed, using 5% of the amount of protein used for DMD cells. Beta-actin was used as a loading control.

Panel D: SD123 fibroblasts from a patient with Schimke Immuno-Osseous Dysplasia, with a homozygous SMARCAL1 nonsense mutation (R17X) were exposed to the indicated concentrations of gentamicin B1 or gentamicin for 6 days and SMARCAL1 levels were determined by western blotting using an anti SMARCAL1 antibody provided by Dr. Cornelius Boerkoel (University of British Columbia). Extracts from WT fibroblasts were also analyzed, using 10% of the amount of protein used for SIOD cells. Beta-actin was used as a loading control.

Panel E: EB14 keratinocytes from a patient with Recessive Dystrophic Epidermolysis Bullosa, with a homozygous Q251X nonsense mutation on the COL7A1 gene were incubated with the indicated concentrations of gentamicin B1 or gentamicin for 72 h and cellular collagen 7 was measured by western blotting using EMD Millipore 234192 collagen 7 antibody. Extracts from WT keratinocytes were also analyzed, using 10% of the amount of protein used for EB14 cells.

### Proposed Synthesis of Compounds

Synthesis of the gentamicin analogues (i.e. see Table B) is proposed via α-glycosylation of the pseudo-disaccharide comprising garosamine linked to deoxystreptamine, either chemically or enzymatically. This would require access to the selectively protected disaccharide in which the alcohol to be glycosylated is free while the other alcohols and amines are protected. One route to this disaccharide would involve first protection of all amines with a suitable blocking group known to one skilled in the art (Cbz, Boc etc), then protection of the syn-diol within the streptamine moiety using Ley's reagent. Subsequent protection of the remaining alcohols and selective removal of the Ley protecting group would leave the pseudo-disaccharide with two free alcohols. Glycosylation of this under conditions for generating 1,2-syn linked product (α-gluco in this case) would likely generate a mixture of the two glycosides from which the one of interest could be separated and protecting groups removed.

Alternatively the direct enzymatic glycosylation of the pseudo-disaccharide comprising garosamine linked to deoxystreptamine may be carried out using a variety of α-glycoside phosphorylases, α-glucosidases (run in trans-glycosylation mode) or available α-glucosyl transferases may prove successful. Large libraries of such enzymes are being assembled making such “screening approaches” feasible. If successful this synthesis may provide a remarkably simple and scalable synthetic route.

### EXAMPLES

### Example 1: p53 Read-Through Assay

Gentamicin, gentamicin A, B, B1, C1, C1a, C2, C2a, C2b, X2, sisomicin, as well as gentamicin fragments garamine and ring C (see FIG. 1) were tested for PTC read-through using the 96-well plate assay. For comparison, G418, a related aminoglycoside that is known to be potent inducer of PTC read-through was used as a positive control. G418 is not an approved drug.

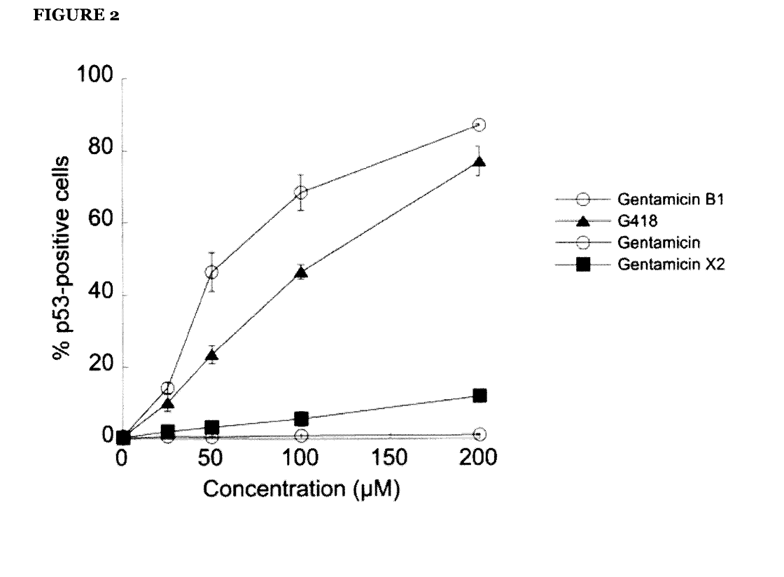

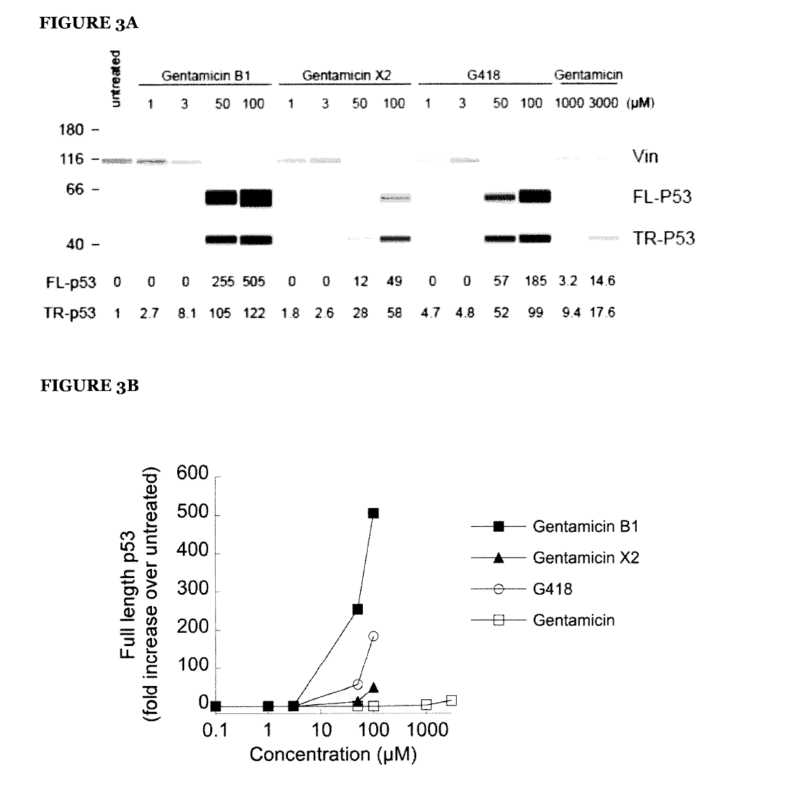

As shown in FIG. 2 Gentamicin did not induce PTC read-through at the concentrations tested, which did not exceed 200 μM. However, it is active at 3 mM as shown in FIG. 3A. Gentamicin A, B, C1, C1a, C2, C2a, C2b, sisomicin, garamine and ring C showed no activity whatsoever (data not shown). G418 showed activity in the 25-200 μM concentration range. Gentamicin X2 showed activity, but it was less potent than G418. Gentamicin B1 showed strong activity, slightly more potent than G418. Therefore, the PTC read-through activity of gentamicin drug is due mostly to the presence of the minor components B1 and X2. Similarly, FIG. 3B shows the induction of PTC read-through by G418, gentamicin, gentamicin B1 and gentamicin X2 using western analysis, wherein the amount of full-length p53 observed in FIG. 3A was plotted versus the concentration of the different compounds on a log scale.

The 96-well plate assay results were confirmed using western analysis as shown in FIG. 3A, wherein HDQ-P1 cells contain very small amounts of p53 protein truncated at R213, and no full-length p53. Induction of PTC read-through causes the appearance of full length p53. Western analysis was performed using an automated quantitative capillary electrophoresis western system. The results confirm the 96-well plate assays and show that gentamicin B1 induces the appearance of full length p53 and that is more potent than G418 or X2. The activity of gentamicin at 3 mM is shown for comparison.

This result is important for medical applications of PTC read-through. Gentamicin is known to be nephrotoxic and ototoxic. (Kohlhepp S. J. et al. 1984) have examined the nephrotoxicity of the major gentamicins C, C1a and C2 and found that nephrotoxicity was caused mainly by C2. Although it is not yet know to what extent gentamicin B1 might be nephrotoxic or ototoxic, it is anticipated that treatment of patients with gentamicin B1 would induce PTC read-through at lower doses than treatment with gentamicin, which typically contains only 0.5-3% B1 (MicroCombiChem™, personal communication). Treatment with gentamicin B1 instead of gentamicin should achieve both higher PTC read-through and lower toxicity via omission of toxic gentamicin C2.

Monitoring of gentamicin plasma concentrations is recommended to avoid toxicity. A cursory search indicates that plasma levels of gentamicin are typically between 1 and 12 μg/ml (2-24 μM) and that concentrations above about 10 μM should be avoided during long-term treatment. The concentrations of gentamicin B1 showing read-through (3 μM and higher) are within this range.

### Example 2: p53 Read-Through Assay with Steroids

As shown in FIG. 4, G418 showed much improved PTC read-through at a concentration of 25 μM in combination with Dexamethasone (5 Betamethazone (5 μM) or Medroxyprogesterone acetate (Medroxy pro)(5 μM), whereas Dexamethasone, Betamethazone alone and Medroxy pro alone showed no read-through activity.

### Example 3: p53 Read-Through Assay with Steroids

The results presented in FIG. 5 show that two gentamicin batches display low PTC readthrough activity at 1 mg/ml while a third batch was inactive (see FIGS. 5A, B, E and F—batch 2). The results also show that gentamicin B1 and gentamicin X2 display potent PTC readthrough activity (see FIGS. 5C-F).

### Example 4: Read-Through Assay Comparing Stop Codons

The results in FIG. 6 show that gentamicin B1 at 50 μg/ml and 100 μg/ml are induce PTC readthrough at all three premature termination codons (i.e. TGA, TAG and TAA). However, there appears to be a slight decrease in readthrough with the TAA stop codon.

### Example 5: Read-Through Assays Comparing Cell Types

FIG. 7 shows that gentamicin B1 can induce PTC readthrough in a variety of cancer cell lines having nonsense mutations at different positions in the TP53 gene (i.e. SW900; NCI-H1688; ESS-1; SK-MES-1; HCCl937; H1299; and HCT116). Gentamicin B1 consistently showed readthrough of the stop codons in various cancer cell lines.

### Example 6: In Vivo Read-Through Assays

As shown in FIG. 8 gentamicin B1 can induce premature termination codon readthrough in a tumour xenograft in vivo. Gentamicin B1 showed readthrough as low as 50 mg/kg (see FIG. 8A), at 200 mg/kg and at 400 mg/kg (see FIG. 8B), whereas no readthrough was detected for gentamicin. No toxicity was observed for B1 but 400 mg/kg gentamicin induced acute toxicity and the mice had to be sacrificed shortly after administration, as denoted by the asterisks.

### Example 7: Induction of PTC Readthrough by Gentamicin B1 in Cells Derived from Patients with Rare Genetic Diseases

FIGS. 9A and B show GM16485 primary fibroblasts derived from a Neuronal Ceroid Lipofuscinosis patient with heterozygous nonsense mutations in the TPP1 (tripeptidylpeptidase I) gene (R127X/R208X) where the fibroblasts were exposed to 25 μg/ml gentamicin B1 or 100 μg/ml gentamicin for up to 10 days and before the fluorescence of cell extracts were measured for TPP1 activity was expressed relative to the average activity of untreated primary fibroblasts from two unaffected individuals (WT) (A). FIG. 9B, shows the same cell extracts analysed for formation of TPP1 by automated capillary electrophoresis western analysis. FIG. 9C shows HSK001 myoblasts derived from a Duchenne Muscular Dystrophy patient with nonsense mutation (DMD: E2035X) were differentiated into myotubes and exposed to the indicated concentrations of gentamicin B1 or gentamicin for 3 days and subsequent dystrophin expression levels were determined by automated capillary electrophoresis western analysis as compared to WT myotubes and loading control. FIG. 9D shows SD123 fibroblasts from a patient with Schimke Immuno-Osseous Dysplasia, with a homozygous SMARCAL1 nonsense mutation (R17X) exposed to the indicated concentrations of gentamicin B1 or gentamicin for 6 days before the SMARCAL1 levels were determined by western blotting as compared to WT fibroblasts and loading control. FIG. 9E shows EB14 keratinocytes from a patient with Recessive Dystrophic Epidermolysis Bullosa, with a homozygous Q251X nonsense mutation on the COL7A1 gene incubated with the indicated concentrations of gentamicin B1 or gentamicin for 72 h prior to cellular collagen 7 measurement by western blotting as compared to WT keratinocytes. In all of the tested genetic diseases gentamicin B1 induced readthrough.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### REFERENCES

Singh A, Ursic D, Davies J. Phenotypic suppression and misreading in Saccharomyces cerevisiae. Nature. 1979; 277:146-8.
Burke J F, Mogg A K Suppression of a nonsense mutation in mammalian cells in vivo by the aminoglycoside antibiotics G418 and paromomycin. Nucl Acids Res. 1985; 13:6265-72.
Bedwell D M, Kaenjak A, Benos D J, Bebok Z, Bubien J K, Hong J, et al. Suppression of a CFTR premature stop mutation in a bronchial epithelial cell line. Nat Med. 1997; 3:1280-4.
Du M, Jones J R, Lanier J, Keeling K M, Lindsey J R, Tousson A, et al. Aminoglycoside suppression of a premature stop mutation in a Cftr−/− mouse carrying a human CFTRG542X transgene. J Mol Med. 2002; 80:595-604.
Clancy J P, Bebök Z, Ruiz F, King C, Jones J, Walker L, et al. Evidence that Systemic Gentamicin Suppresses Premature Stop Mutations in Patients with Cystic Fibrosis. Am J Respir Crit Care Med. 2001; 163:1683-92.
Wilschanski M, Yahav Y, Yaacov Y, Blau H, Bentur L, Rivlin J, et al. Gentamicin-induced correction of CFTR function in patients with cystic fibrosis and CFTR stop mutations. N Engl J Med. 2003; 349:1433-41.
Linde L, Boelz S, Nissim-Rafinia M, Oren Y S, Wilschanski M, Yaacov Y, et al. Nonsensemediated mRNA decay affects nonsense transcript levels and governs response of cystic fibrosis patients to gentamicin. J Clin Invest. 2007; 117:683-92.
Barton-Davis E R, Cordier L, Shoturma D I, Leland S E, Sweeney H L. Aminoglycoside antibiotics restore dystrophin function to skeletal muscles of mdx mice. J Clin Invest. 1999; 104:375-81.
Malik V, Rodino-Klapac L R, Viollet L, Wall C, King W, Al-Dahhak R, et al. Gentamicin induced readthrough of stop codons in Duchenne muscular dystrophy. Ann Neurol. 2010; 67:771-80.
Shulman E, Belakhov V, Wei G, Kendall A, Meyron-Holtz E G, Ben-Shachar D, et al. Designer aminoglycosides that selectively inhibit cytoplasmic rather than mitochondrial ribosomes show decreased ototoxicity: a strategy for the treatment of genetic diseases. J Biol Chem. 2014; 289:2318-30.
Xue X, Mutyam V, Tang L, Biswas S, Du M, Jackson L a, et al. Synthetic aminoglycosides efficiently suppress cystic fibrosis transmembrane conductance regulator nonsense mutations and are enhanced by ivacaftor. Am J Respir Cell Mol Biol. 2014; 50:805-16.
Gatti R A. SMRT compounds correct nonsense mutations in primary immunodeficiency and other genetic models. Ann N Y Acad Sci. 2012; 1250:33-40.
Welch E M, Barton E R, Zhuo J, Tomizawa Y, Friesen W J, Trifillis P, et al. PTC124 targets genetic disorders caused by nonsense mutations. Nature. 2007; 447:87-91.
McElroy S P, Nomura T, Torrie L S, Warbrick E, Gartner U, Wood G, et al. A lack of premature termination codon read-through efficacy of PTC124 (Ataluren) in a diverse array of reporter assays. PLoS Biol. 2013; 11:e1001593.
Du M, Liu X, Welch E M, Hirawat S, Peltz S W, Bedwell D M. PTC124 is an orally bioavailable compound that promotes suppression of the human CFTR-G542X nonsense allele in a C F mouse model. Proc Natl Acad Sci USA. 2008; 105:2064-9.
Kerem E. Ataluren for the treatment of nonsense-mutation cystic fibrosis: a randomised, double-blind, placebo-controlled phase 3 trial. Lancet Respir Med. 2014; 18:11-2.
Bushby K, Finkel R, Wong B, Barohn R, Campbell C, Comi G P, et al. Ataluren treatment of patients with nonsense mutation dystrophinopathy. Muscle Nerve. 2014; 50:477-87.
Ryan N J. Ataluren: first global approval. Drugs. 2014; 74:1709-14.
Karijolich J, and Yu, Y-T, Therapeutic suppression of premature termination codons: Mechanisms and clinical considerations (Review) Int J Mol Med 2014; 34:355-362.
Mort, M., Ivanov, D., Cooper, D. N. and Chuzhanova N. A. A Meta-Analysis of Nonsense Mutations Causing Human Genetic Disease. Human Mutation 2008; 29(8):1037-1047.
Kohlhepp S. J., et al. Nephrotoxicity of the constituents of the gentamicin complex. J. Infectious Diseases 1984; 149(4):605-614.
Wang C. S. et al. Establishment and characterization of a new cell line derived from a human primary breast carcinoma. Cancer Genet Cytogenet. 2000; 120(1):58-72.
Floquet C, Deforges J, Rousset J-P, Bidou L. Rescue of non-sense mutated p53 tumor suppressor gene by aminoglycosides. Nucleic Acids Res. 2011; 39:3350-3362.
Floquet C. et al. Statistical analysis of readthrough levels for nonsense mutations in mammalian cells reveals a major determinant of response to gentamicin. PLoS Genet. 2012; 8(3):e1002608.
Liang, S. H. and Clarke M. F. Regulation of p53 localization. Eur J Biochem. 2001; 268(10):2779-2783.
Shaulsky, G. et al. Nuclear accumulation of p53 protein is mediated by several nuclear localization signals and plays a role in tumorigenesis. Mol Cell Biol. 1990; (12):6565-6577.
Keeling, K. M. et al. Therapeutics Based on Stop Codon Readthrough. Annu. Rev. Genomics Hum. Genet. 2014; 15:8.1-8.24.
Frischmeyer P A, Dietz H C: Nonsense-mediated mRNA decay in health and disease. Hum Mol Genet 1999, 8(10):1893-1900.
Chang Y F, Imam J S, Wilkinson M F: The nonsense-mediated decay RNA surveillance pathway. Annu Rev Biochem 2007, 76:51-74.
Isken O, Maquat L E: Quality control of eukaryotic mRNA: safeguarding cells from abnormal mRNA function. Genes Dev 2007, 21(15):1833-1856.
Rebbapragada I, Lykke-Andersen J: Execution of nonsense-mediated mRNA decay: what defines a substrate? Curr Opin Cell Biol 2009, 21(3):394-402.
Rehwinkel J, Raes J, Izaurralde E: Nonsense-mediated mRNA decay: Target genes and functional diversification of effectors. Trends Biochem Sci 2006, 31(11):639-646.
Muhlemann O, Eberle A B, Stalder L: Zamudio Orozco R: Recognition and elimination of nonsense mRNA. Biochim Biophys Acta 2008, 1779 (9):538-549.
Hoe, K. K. Verma, C. S. and Lane, D. P. NATURE REVIEWS I DRUG DISCOVERY MARCH (2014) 13:217-236.
Petitjean A, Mathe E, Kato S, Ishioka C, Sean V, Hainaut P, et al. Impact of Mutant p53 Functional Properties on TP53 Mutation Patterns and Tumor Phenotype: Lessons from Recent Developments in the IARC TP53 Database. Hum Mutat. 2007; 28:622-9.
Vogelstein B, Papadopoulos N, Velculescu V E, Zhou S, Jr LAD, Kinzler K W. Cancer Genome Landscapes. Science (80-). 2013; 339:1546-58.
Baradaran-Heravi, A., Balgi, A. D., Zimmerman, C., Choi, K., Shidmoosavee, F. S., Tan, J. S., Bergeaud, C., Krause, A., Flibotte, S., Shimizu, Y., Anderson, H. J., Jan, E., Pfeifer, T.,
Jaquith, J. B., Roberge, M. Novel small molecules potentiate premature termination codon readthrough by aminoglycosides. Nucleic Acids Res. (2016) 44: 6538-6598.
Lojewski, X., Staropoli, J. F., Biswas-Legrand, S. et al. (2014) Human iPSC models of neuronal ceroid lipofuscinosis capture distinct effects of TPP1 and CLN3 mutations on the endocytic pathway. Hum. Mol. Genet. (2014) 23: 2005-2022.

1.-13. (canceled)

14. A method of treating or ameliorating a medical condition associated with premature termination codons (PTCs) in RNA, the method comprising administering a compound, or a pharmaceutically acceptable salt thereof, in an amount effective for treating or ameliorating a medical condition associated with a PTC in RNA, wherein the compound has the structure of Formula I: 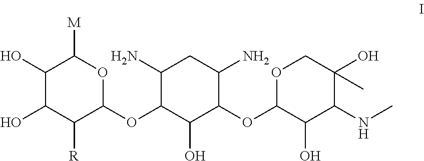
wherein
R is OH or NH₂;
M is 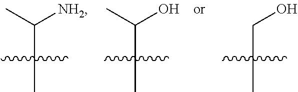
when R is OH and M is 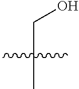 [SMILES: *CO]
when R is NH₂;
to a subject in need thereof.

15. The method of claim 14, wherein the compound selected from one or more of the following: 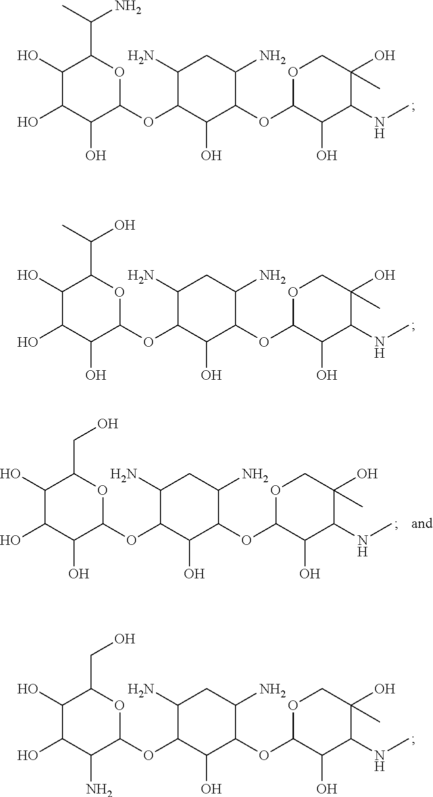 [SMILES: CNC1C(O)C(OC2C(N)CC(N)C(OC3OC(C(C)N)C(O)C(O)C3O)C2O)OCC1(C)O ; CNC1C(O)C(OC2C(N)CC(N)C(OC3OC(C(C)O)C(O)C(O)C3O)C2O)OCC1(C)O ; CNC1C(O)C(OC2C(N)CC(N)C(OC3OC(CO)C(O)C(O)C3O)C2O)OCC1(C)O ; CNC1C(O)C(OC2C(N)CC(N)C(OC3OC(CO)C(O)C(O)C3N)C2O)OCC1(C)O]
or the pharmaceutical composition thereof.

16. The method of claim 14, wherein the compound is selected from one or more of the following:

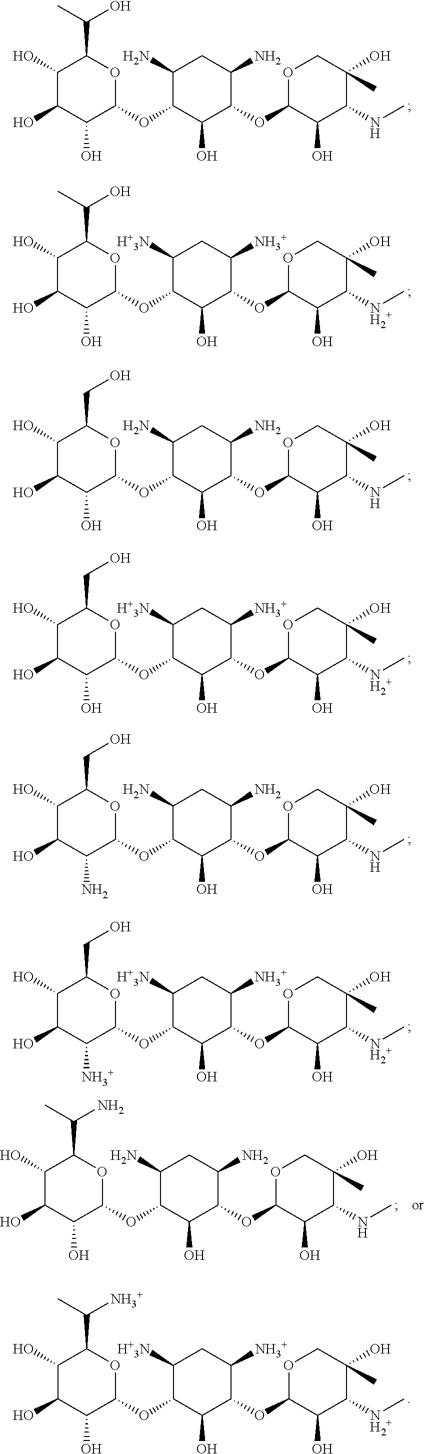

17. The method of claim 14, wherein the medical condition is selected from TABLE 1 or TABLE 2.

18. The method of claim 14, wherein the medical condition is selected from the group consisting of: central nervous system disease; peripheral nervous system disease; neurodegenerative disease; autoimmune disease; DNA repair disease; inflammatory disease; collagen disease; kidney disease; pulmonary disease; eye disease; cardiovascular disease; blood disease; metabolic disease; neuromuscular diseases; neoplastic disease; and any genetic disorder caused by nonsense mutation(s).

19. The method of claim 18, wherein the medical condition is selected from the group consisting of: ataxia-telangiectasia; muscular dystrophy; Duchenne muscular dystrophy; Dravet syndrome; myotonic dystrophy; multiple sclerosis; infantile neuronal ceroid lipofuscinosis; Alzheimer's disease; Tay-Sachs disease; neural tissue degeneration; Parkinson's disease; chronic rheumatoid arthritis; lupus erythematosus; graft-versus-host disease; primary immunodeficiencies; severe combined immunodeficiency; DNA Ligase IV deficiency; Nijmegen breakage disorders; xeroderma pigmentosum (XP); rheumatoid arthritis; hemophilia; von Willebrand disease; thalassemia (for example; β-thalassemia); familial erythrocytosis; nephrolithiasis; osteogenesis imperfecta; cirrhosis; neurofibroma; bullous disease; lysosomal storage diseases; Hurler's disease; familial cholesterolemia; cerebellar ataxia; tuberous sclerosis; immune deficiency; cystic fibrosis; familial hypercholesterolemia; pigmentary retinopathy; retinitis pigmentosa; amyloidosis; atherosclerosis; giantism; dwarfism; hypothyroidism; hyperthyroidism; aging; obesity; diabetes mellitus; familial polycythemia; Niemann-Pick disease; epidermolysis bullosa; Marfan syndrome; Becker muscular dystrophy (BMD); spinal muscular atrophy; cancer; and any genetic disorder caused by nonsense mutation(s).

20. The method of claim 19, wherein the cancer is of the head and neck, eye, skin, mouth, throat, esophagus, chest, bone, blood, lung, colon, sigmoid, rectum, stomach, prostate, breast, ovaries, kidney, liver, pancreas, brain, intestine, heart or adrenals.

21. The method of claim 19, wherein the cancer is sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, Kaposi's sarcoma, pinealoma, hemangioblastoma, acoustic neuroma, oligodendroglioma, menangioma, melanoma, neuroblastoma, retinoblastoma, a blood-born tumor or multiple myeloma.

22. The method of claim 19, wherein the cancer is acute lymphoblastic leukemia, acute lymphoblastic B-cell leukemia, acute lymphoblastic T-cell leukemia, acute myeloblastic leukemia, acute promyelocytic leukemia, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia, chronic lymphocytic leukemia, hairy cell leukemia, or multiple myeloma.

23. The method of claim 14, wherein the premature termination codon is UGA or UAG.

24. The method of claim 14, wherein the premature termination codon is UGA.

25. The method of claim 14, wherein the premature termination codon is UAG.

26. The method of claim 14, wherein the premature termination codon is UAA.

27.-28. (canceled)

29. A compound, wherein the compound has the structure:

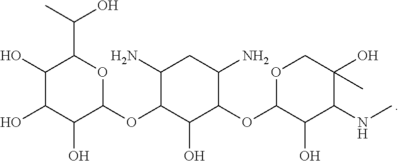 [SMILES: CNC1C(O)C(OC2C(N)CC(N)C(OC3OC(C(C)O)C(O)C(O)C3O)C2O)OCC1(C)O]

30.-60. (canceled)
